(19) United States
(12) Patent Application Publication
(10) Pub. No.: US 2013/0078673 A1
(43) Pub. Date: Mar. 28, 2013

(54) RECOMBINANT MICROORGANISM HAVING AN ABILITY OF USING SUCROSE AS A CARBON SOURCE

(71) Applicant: KOREA ADVANCED INSTITUTE OF SCIENCE A, Daejeon (KR)

(72) Inventors: Sang Yup LEE, Daejeon (KR); Jeong Wook LEE, Daejeon (KR); Hyohak SONG, Daejeon (KR); Ji Mahn KIM, Seoul (KR); Sol CHOI, Jeju-do (KR); Jin Hwan PARK, Gyeonggi-do (KR)

(73) Assignee: KOREA ADVANCED INSTITUTE OF SCIENCE AND TECHNOLOGY, Daejeon (KR)

(21) Appl. No.: 13/667,612

(22) Filed: Nov. 2, 2012

Related U.S. Application Data
(62) Division of application No. 12/808,533, filed on Jun. 20, 2011.

(30) Foreign Application Priority Data
Dec. 18, 2007 (KR) 10-2007-0133239
Oct. 28, 2008 (KR) 10-2008-0106113

(51) Int. Cl. C12N 15/70

(52) U.S. Cl. CPC C12N 15/70
USPC 435/69.1; 435/320.1; 435/252.3; 435/254.2; 435/254.11; 435/105

(57) ABSTRACT

The present invention relates to a recombinant microorganism capable of metabolizing sucrose, and more particularly to a recombinant microorganism capable of metabolizing sucrose in which a gene encoding sucrose phosphotransferase and/or a gene encoding sucrose-6-phosphate hydrolase is introduced or to a recombinant microorganism capable of metabolizing sucrose in which a gene encoding β-fructofuranosidase is introduced. According to the present invention, a recombinant microorganism capable of using inexpensive sucrose as a carbon source instead of expensive glucose is provided. In addition, in a process of culturing microorganisms which have been incapable of using sucrose as a carbon source, sucrose can substitute for other carbon sources including glucose.

### TECHNICAL FIELD

The present invention relates to a recombinant microorganism capable of metabolizing sucrose, and more particularly to a recombinant microorganism capable of metabolizing sucrose in which a gene encoding sucrose phosphotransferase and/or a gene encoding sucrose-6-phosphate hydrolase is introduced or to a recombinant microorganism capable of metabolizing sucrose in which a gene encoding β-fructofuranosidase is introduced.

### BACKGROUND ART

For the sustainable development of mankind, studies for the development of the industrial biotechnology for the production of useful compounds from renewable bio-resources are being actively conducted along with a great interest therein. The production of chemical substances by microbial fermentation has been performed to date using glucose as a main raw material. However, the production of chemical products by microbial fermentation is difficult to commercialize, because glucose is expensive and thus the price of chemical compounds produced by the microbial fermentation is higher than that of chemical compounds produced by chemical synthetic methods that use crude oil as a main raw material. Thus, to develop as an alternative to the method that uses expensive glucose as a carbon source, studies on the discovery of a variety of inexpensive carbon sources which can be easily obtained from abundant bioresources are being actively conducted. For example, studies on the production of various primary metabolites using relatively inexpensive raw materials, such as lignocellulosic hydrolysates, glycerol, whey, corn steep liquors or the like, have been conducted by many researchers including the present inventors (Samuelov et al., Appl. Environ. Microbiol., 65:2260, 1999; Lee et al., Appl. Microbiol. Biotechnol., 54:23, 2000; Lee et al., Biotechnol. Bioeng., 72:41, 2001; Lee et al., Biotechnol. Lett., 25:111, 2003; Lee et al., Bioproc. Biosystems Eng., 26:63, 2003). However, according to the study results reported to date, when the raw materials were used as carbon sources instead of glucose, the productivity or production yield of desired metabolites was significantly lower than when glucose was used as a carbon source. Thus, research and development are urgently required to overcome this drawback.

Sucrose (commonly known as sugar) is a disaccharide consisting of glucose and fructose, and it is a carbon source that is very abundant in nature and is produced from all plants having photosynthesis ability. Particularly, sugarcane and sugar beet contain large amounts of sucrose, and more than 60% of the world's sucrose is currently being produced from sugarcane. Particularly, sucrose is produced at a very low cost, because it can be industrially produced through a simple process of evaporating/concentrating extracts obtained by mechanical pressing of sugarcanes. Koutinas et al. calculated the prices of various raw materials usable for the microbial production of chemical substances on the basis of the glucose contents in the year 2004 and, as a result, the price of sucrose based on 1 kg of the glucose content was 26.1 cents which is a very low price corresponding to 77% of the wheat price, 50% of the molasses price and 28.9% of the sucrose price (Koutinas et al., Ind. Crops and Products, 20:75, 2004).

A report on the International Sugar Agreement (ISA) daily price indicates that the price of sucrose is on a steady downward trend after peaking in 1994-1995 due to surplus supply and that the downward trend is expected to continue. Accordingly, sucrose is receiving attention as the most potent carbon source which will substitute for expensive glucose that is currently being used to produce various chemical compounds through microbial fermentation. Particularly, it is well known to those skilled in the art to it is very difficult to reduce the production cost of glucose to the level of sucrose, because glucose that is produced mainly from corn starch is produced through very complicated processes including extraction of starch from corn, thermal/chemical pretreatment of starch, conversion of starch to glucose by enzymatic reactions, and purification of glucose, and because the price of corn is continuously increasing. For these reasons, corn-based bioethanol production in the USA is gradually decreasing (Mae-Wan Ho, Science in Society, 33:40, 2007), but sugarcane (i.e., sucrose)-based bioethanol production in Brazil is rapidly growing.

To date, studies on the production of useful compounds using sucrose as a carbon source have been conducted with respect to the production of biodegradable polymers (Lee et al., Biotechnol. Lett., 15:971, 1993; Lee et al., Biotechnol. Techniques, 1:59, 1997), citric acid (Forster et al., App. Microbiol. Biotechnol., 75:1409, 2007), acetone, butanol, ethanol and isopropanol (George et al., Appl. Environ. Microbiol., 45:1160, 1983; Durre, Appl. Microbiol. Biotechnol., 49:639, 1998), itaconic acid (Kautola et al., Biotechnol. Lett., 11:313, 1989), xanthan gum (Letisse et al., Appl. Microbiol. Biotechnol., 55:417, 2001), etc., by high-concentration cell culture. Particularly, the report (2006) of the International Energy Agency (IEA) Bioenergy Task 40, which analyzes international bioenergy and biofuel trade evaluated that bioethanol production from sugarcane (including sucrose) in Brazil is an excellent model of environmentally friendly, sustainable biofuel production.

Sucrose as a raw material for producing chemical compounds through microbial fermentation is inexpensive and can function to protect the cell membrane from an external environment containing large amounts of desired metabolites, thus producing high-concentrations of desired metabolites. Kilimann et al. exposed microorganisms to a medium containing sucrose and a medium containing no sucrose at lethal temperatures and then examined the viability thereof and the secondary structures of the proteins (Biochimica et Biophysica Acta, 1764, 2006).

The study results revealed that the secondary structures of proteins in the cells of the microorganisms exposed to the medium containing sucrose were very well conserved, but the structures of proteins in the cells of the microorganisms exposed to the medium containing sucrose were highly modified. Particularly, the viability of the microorganisms exposed to the medium containing sucrose was significantly higher than that of the microorganisms exposed to the medium containing no sucrose. This directly demonstrates the function of sucrose to protect microorganisms from a harmful external environment.

Even though sucrose is an excellent raw material having the above-described advantages, including low price and a function to protect microorganisms from an external environment, an example showing the successful production of desired chemical compounds using sucrose as a carbon source and the actual commercial application thereof has not yet been reported. This is because a large number of microorganisms do not have a complete mechanism of transporting sucrose into cells, degrading the transported sucrose and linking the degraded products to glycolysis, and thus cannot use sucrose as a carbon source. Even in the case of microorganisms having a mechanism capable of using sucrose, they cannot efficiently produce desired metabolites, because the rate of ingestion and degradation of sucrose as a carbon source is very low.

In order for the production of various chemical compounds through microbial fermentation to be performed in an industrially economic manner, the use of an inexpensive raw material such as sucrose as described above is required, and furthermore, the identification of an enzyme capable of efficiently ingesting and degrading sucrose as a carbon source at a high rate and the research and development enabling the use of the enzyme are necessarily required. Particularly, in view of the fact that the price of raw materials for producing chemical compounds through microbial fermentation account for about 50% of the price of final products, the identification of an enzyme enabling efficient use of sucrose as an inexpensive raw material and the development of the application of the enzyme are urgently required.

It has been reported that sucrose can be used for producing various bioproducts, including biodegradable polymers, citric acid, itaconic acid, acetone, butanol, ethanol, isopropanol and xanthan gum, by high-concentration cell culture. However, examples showing the successful production of desired chemical compounds through microbial fermentation and the actual commercial application thereof have been rarely reported.

Thus, in order for the production of various chemical compounds through microbial fermentation as an environmentally friendly technology to be successfully applied in the industry, the development of microorganisms capable of effectively ingesting and degrading an inexpensive and abundant carbon source such as sucrose is required.

### SUMMARY OF INVENTION

It is, therefore, an object of the present invention to provide novel sucrose metabolism-related genes enabling sucrose to be used as a carbon source, and enzymes which are encoded by the genes.

Another object of the present invention is to provide a recombinant microorganism capable of metabolizing sucrose in which the sucrose metabolism-related gene is introduced, and a method for producing metabolites, biodegradable polymer or recombinant proteins using the recombinant microorganism.

In order to achieve the above objects, the present invention provides a sucrose phosphotransferase having an amino acid sequence of SEQ ID NO: 1; a gene (ptsG) encoding said sucrose phosphotransferase; and a recombinant vector containing said gene (ptsG) and a gene (sacC) encoding a sucrose-6-phosphate hydrolase.

The present invention also provides a recombinant microorganism capable of metabolizing sucrose in which said gene is introduced into a host cell selected form the group consisting of bacteria, yeast and fungi; and a method for producing metabolites, biodegradable polymers or recombinant proteins, the method comprises culturing said recombinant microorganism in a medium containing sucrose as a carbon source.

The present invention also provides a β-fructofuranosidase having an activity to hydrolyze β-D-fructofuranoside bond to liberate fructose; and a gene encoding said β-fructofuranosidase.

The present invention also provides a recombinant microorganism capable of metabolizing sucrose in which said gene is introduced into a host cell selected form the group consisting of bacteria, yeast and fungi; and a method for producing metabolites, biodegradable polymers or recombinant proteins, the method comprises culturing said recombinant microorganism in a medium containing sucrose as a carbon source.

Other features and aspects of the present invention will be more apparent from the following detailed description and the appended claims.

### BRIEF DESCRIPTION OF DRAWINGS

FIG. 1 is a schematic diagram showing a pathway through which sucrose is ingested and degraded, and the degraded products are metabolized through glycolysis, when novel metabolism-related enzymes (sucrose phosphotransferase, sucrose-6-phosphate hydrolase, and fructokinase) derived from M. succiniciproducens MBEL55E, which enable the metabolism of sucrose, are introduced into a microorganism incapable of metabolizing sucrose. Thick arrows (→): indicates metabolic pathways in which the introduced novel sucrose metabolism-related enzymes derived from M. succiniciproducens MBEL55E are involved; and thin arrows (→): indicate the original metabolic pathways of the recombinant microorganism.

FIG. 2 is a schematic diagram showing four possible pathways how a novel β-fructofuranosidase derived from M. succiniciproducens MBEL55E, which enables the metabolism of sucrose, can be involved in sucrose metabolism, when the enzyme is introduced into a microorganism incapable of metabolizing sucrose. Thick arrows (→): four possible pathways in which the introduced novel β-fructofuranosidase derived from M. succiniciproducens MBEL55E will be involved; and thin arrows (→): indicate the original metabolic pathways of the recombinant microorganism.

FIG. 3 is a map of recombinant vector pMSscrIIA containing genes (ptsG, sacC and rbsK) encoding sucrose phosphotransferase, sucrose-6-phosphate hydrolase and fructokinase.

FIG. 4 is a graphic diagram showing the growth of parent strain MBEL55E, a recombinant MptsG strain and a recombinant MsacC strain in sucrose media (: MBEL55E; ▴: MptsG; and Δ: MsacC).

FIG. 5 is a cleavage map of recombinant vector pTac15K.

FIG. 6 is a cleavage map of recombinant vector pTac15KsacC containing a gene encoding sucrose-6-phosphate hydrolase.

FIG. 7 is a graphic diagram showing the growth of recombinant E. coli W3110 pTac15K in a M9 medium containing sucrose (solid line including : sucrose concentration; and solid line including □: OD₆₀₀).

FIG. 8 is a graphic diagram showing the growth of the inventive recombinant E. coli W3110 pTac15KsacC, having the ability to metabolize sucrose, in a M9 medium containing sucrose (solid line including : sucrose concentration; solid line including □: OD₆₀₀; dot line including : glucose concentration; solid line including ◯: fructose concentration; and dot line including ▾: acetic acid concentration).

FIG. 9 is a graphic diagram showing the growth of E. coli W3110 pTac15KEWcscA and E. coli W3110 pTac15 KBSsacA in M9 media containing sucrose (solid line including : E. coli W3110 pTac15KEWcscA; and solid line including ▴: E. coli W3110 pTac15 KBSsacA).

FIG. 10 is a graphic diagram showing the growth and metabolite production of the inventive E. coli W3110 pTac15KsacC capable of metabolizing sucrose, fermented in anaerobic conditions (solid line including : sucrose concentration; solid line including □: OD₆₀₀; solid line including ▾: glucose concentration; solid line including ▪: fructose concentration; dot line including : succinic acid concentration; dot line including □: lactic acid concentration; dot line including ▪: formic acid concentration; dot line including □: acetic acid concentration; and dot line including ▴: ethanol concentration).

### DETAILED DESCRIPTION OF THE INVENTION AND PREFERRED EMBODIMENTS

The present invention aims at the production of bioproducts using sucrose (commonly known as sugar) as an inexpensive and abundant carbon source, particularly the discovery of either a microorganism capable of using sucrose as a carbon source or enzymes enabling the use of a microorganism incapable of using sucrose, and aims to use the enzymes to produce bioproducts.

Sucrose which is a disaccharide consisting of glucose and fructose is a globally abundant carbon source. It is produced from most plants having photosynthesis ability, and particularly, sugarcane and sugar beet which are tropical crops and subtropical crops contain large amounts of sucrose. Sucrose can be industrially produced through a simple process of evaporating and concentrating an extract obtained by mechanical pressing of sugarcane, and more than 60% of the world's sucrose is currently being produced from sugarcane. According to the paper (published in 2004) of Koutinas et al. who calculated the prices of various raw materials usable in microbial fermentation on the basis of the glucose contents, the price of sucrose based on 1 kg of the glucose content is 26.1 cents which is ¼ of the glucose price and ½ to ⅔ of the wheat and molasses prices (Koutinas et al., Ind. Crops and Products, 20:75, 2004). Furthermore, with respect to the change in the sucrose price published in the International Sugar Organization for recent 15 years, the sucrose price declined sharply to 6.27 cents/lb in 1999 after peaking to 13.28 cents/lb in 1995, increased again to 14.20 cents/lb on March, 2008 and was maintained at a level of 12.44 cents/lb on 7 May, 2008, and the sucrose price is expected to be on a downward trend due to surplus supply. In addition to this advantage in terms of cost, sucrose is advantageous in that it can be relatively stably supplied even in food crisis circumstances such as recent grain crisis, because it is not obtained from grain.

To date, it has been reported that sucrose can be used for producing various bioproducts, including biodegradable polymers, citric acid, itaconic acid, acetone, butanol, ethanol, isopropanol and xanthan gum (Lee et al., Biotechnol. Lett., 15:971, 1993; Lee et al., Biotechnol. Techniques, 1:59, 1997; Forster et al., App. Microbiol. Biotechnol., 75:1409, 2007; George et al., Appl. Environ. Microbiol., 45:1160, 1983; Durre, Appl. Microbiol. Biotechnol., 49:639, 1998; Kautola et al., Biotechnol. Lett., 11:313, 1989; Letisse et al., Appl. Microbiol. Biotechnol., 55:417, 2001), by high-concentration cell culture. This suggests that an improvement in the utility of sucrose can have a direct influence on the effective production of bioproducts.

However, despite several advantages of sucrose, including advantages as a raw materials, a function to protect proteins from modification, and a function to protect cells from external environments (Kilimann et al., Biochimica et Biophysica Acta, 1764, 2006), examples showing the successful production of desired chemical compounds through microbial fermentation using sucrose as a carbon source and the actual commercial application thereof have been rarely reported. One reason therefor is that a large number of microorganisms cannot effectively produce desired bioproducts, because the microorganisms do not have a mechanism capable of metabolizing sucrose or have a slow metabolic rate, even if they have the mechanism. Thus, in order for the production of various chemical compounds through microbial fermentation as an environmentally friendly technology to be successfully applied in the industry, the development of microorganisms capable of effectively ingesting and degrading an inexpensive and abundant carbon source such as sucrose is required. For this purpose, the identification of an enzyme capable of degrading and metabolizing at high rate and the utilization thereof must be performed.

To date, an enzyme group enabling the use of sucrose has been developed by several researchers. Typical examples include the technology of Ajinomoto Co. in which E. coli-derived PTS (phosphoenol pyruvate-dependent phosphotransferase system) and a sucrose transport system of non-PTS are introduced and used to produce amino acids. This technology has a characterized in that a whole gene group associated with PTS or non-PTS is introduced, thus completing an invention.

However, in the present invention, based on the genetic information of Mannheimia succiniciproducens MBEL55E (KCTC 0769BP), novel genes (ptsG, sacC and rbsK) encoding enzymes [sucrose phosphotransferase (PtsG, MS0784), sucrose-6-phosphate hydrolase (SacC, MS0909) and fructokinase (RbsK, MS1233)] which are involved in transporting sucrose into cells, degrading the transported sucrose and linking the degraded products to glycolysis were found, and the sequences and functions thereof were identified.

Accordingly, in one aspect, the present invention relates to sucrose phosphotransferase having an amino acid sequence of SEQ ID NO: 1, sucrose-6-phosphate hydrolase having an amino acid sequence of SEQ ID NO: 3, and fructokinase having an amino acid sequence of SEQ ID NO: 5, which are enzymes that are involved in transporting sucrose into cells, degrading the transported sucrose and linking the degraded products to glycolysis. The present invention also relates to a gene (ptsG) encoding said sucrose phosphotransferase, a gene (sacC) encoding said sucrose-6-phosphate hydrolase, and a gene (rbsK) encoding said fructokinase.

In the present invention, the sucrose phosphotransferse (PtsG) functions to transport sucrose into cells while converting sucrose into sucrose-6-phosphate, the sucrose-6-phosphate hydrolase (SacC) has an activity to convert sucrose-6-phosphate to glucose-6-phosphate and fructose, and the fructokinase (RbsK) has an activity to convert fructose to fructose-6-phosphate.

In the present invention, the ptsG is preferably represented by a base sequence of SEQ ID NO: 2, the sacC is preferably represented by a base sequence of SEQ ID NO: 4, and rbsK is represented by a base sequence of SEQ ID NO: 6.

Specifically, in the present invention, a recombinant vector containing the ptsG and sacC genes was constructed, and then introduced into E. coli incapable of using sucrose as a carbon source, and the constructed recombinant E. coli was cultured in a medium containing sucrose as a single carbon source. As a result, it was found that the recombinant E. coli had the ability to metabolite sucrose.

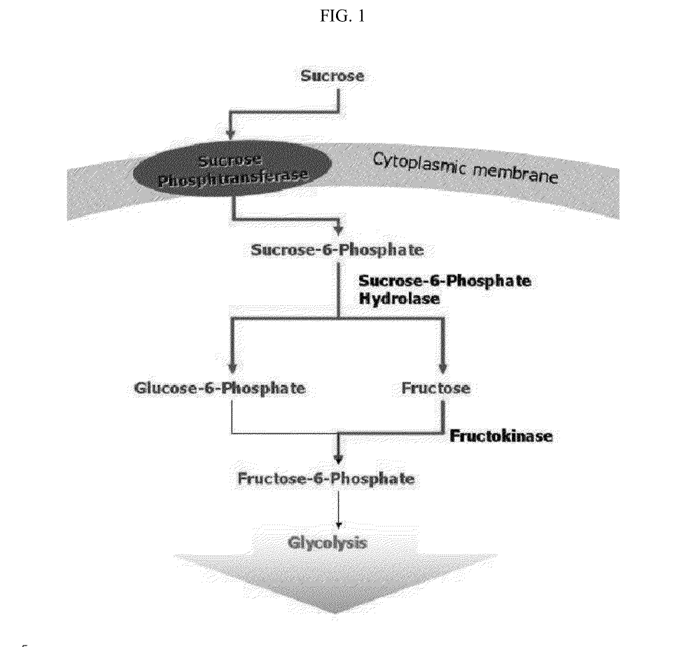

Accordingly, as shown in FIG. 1, it can be inferred that sucrose is transported into cells by the sucrose phosphotransferase (PtsG) while being converted to sucrose-6-phosphate, the sucrose-6-phosphate transported into cells is converted to glucose-6-phosphate and fructose by the sucrose-6-phosphate hydrolase (SacC), the fructose is converted to fructose-6-phosphate by the fructokinase (RbsK), and the degraded product are linked to glycolysis.

Meanwhile, the present inventors have demonstrated that microorganisms which have not been capable of metabolizing sucrose can metabolize sucrose by the introduction of sucrose-6-phosphate hydrolase (SacC, MS0909) that is β-fructofuranosidase derived from Mannheimia succiniciproducens MBEL55E (KCTC 0769BP), as well as other novel genes (cscA and sacA) encoding β-fructofuranosidase, and have demonstrated examples of producing various metabolites using the microbial strain, thereby completing the present invention. In other words, based on the genetic information of Mannheimia succiniciproducens MBEL55E (KCTC 0769BP), E. coli W and Bacillus subtilis, novel genes (sacC, cscA, and sacA) encoding β-fructofuranosidase that is an enzyme involved in degrading sucrose and linking the degraded products to glycolysis were discovered, and the sequences and functions thereof were identified. EC number (Enzyme Commission number) provided by the Nomenclature Committee of the International Union of Biochemistry and Molecular Biology (http://www.iubmb.org/) is a well-known numerical classification scheme for enzymes, based on the chemical reactions they catalyze.

The official name for sucrose-6-phosphate hydrolase is β-fructofuranosidase, (EC 3.2.1.26)) and has other names including β-D-fructofuranoside fructohydrolase, invertase, saccharase, glucosucrase, β-h-fructosidase, β-fructosidase, invertin, sucrase, maxinvert L 1000, fructosylinvertase, alkaline invertase, acid invertase (http://www.chem.qmul.ac.uk/iubmb/enzyme/EC3/2/1/26.html). Namely, sucrose-6-phosphate hydrolase and sucrase all have the official name β-fructofuranosidase (EC 3.2.1.26).

Such β-fructofuranosidase catalyzes the hydrolysis of terminal non-reducing β-D-fructofuranoside residues in β-D-fructofuranoside. It has the official name “sucrase or invertase” when it catalyzes the hydrolysis of sucrose, and has the official name “sucrose-6-phosphate hydrolase” when it catalyzes the hydrolysis of sucrose-6-phosphate.

In Examples of the present invention, in addition to demonstrating the sucrose-metabolizing ability caused by the introduction of sucrose-6-phosphate hydrolase (EC 3.2.1.26, SacC) derived from Mannheimia, that is, β-fructofuranosidase, an attempt was made to demonstrate the introduction of general β-fructofuranosidase into microorganisms imparts the sucrose-metabolizing ability to the microorganisms. As a result, when each of E. coli W-derived invertase (EC 3.2.1.26, CscA) and Bacillus subtilis-derived β-fructofuranosidase was introduced into microorganisms incapable of metabolizing sucrose, it was observed that the microorganisms introduced with β-fructofuranosidase (EC 3.2.1.26) grew by metabolizing sucrose.

Accordingly, in another aspect, the present invention relates to β-fructofuranosidase having activities to hydrolyze β-D-fructofuranoside bond to liberate fructose, including an activity to hydrolyze sucrose to glucose and fructose and an activity to hydrolyze sucrose-6-phosphate to glucose-6-phosphate and fructose. Also, the β-fructofuranosidase may have an amino acid sequence selected from the group consisting of amino acid sequences of SEQ ID NO: 3, SEQ ID NO: and SEQ ID NO: 9, but the scope of the present invention is not limited thereto.

Specifically, in the present invention, sucrose-6-phosphate hydrolase (SacC) that is one example of the β-fructofuranosidase has an activity to convert sucrose-6-phosphate to glucose-6-phosphate and fructose or an activity to convert sucrose to glucose and fructose. Also, in Examples of the present invention, β-fructofuranosidase derived from Mannheimia was used, but those derived from other microorganisms fall within the scope of the present invention and may have an amino acid sequence of SEQ ID NO: 3, and a gene (sacC) encoding the same is preferably represented by a base sequence of SEQ ID NO: 4.

In the present invention, invertase, sucrase and sucrose hydrolase (CscA) that are examples of the β-fructofuranosidase are enzymes which are involved in degrading sucrose and linking the degraded products to glycolysis, and these enzymes have an activity to convert sucrose-6-phosphate to glucose-6-phosphate and fructose or an activity to convert sucrose to glucose and fructose. Furthermore, in Examples of the present invention, the β-fructofuranosidase derived from E. coli W was illustrated, but those derived from other microorganisms also fall within the scope of the present invention and may have an amino acid sequence of SEQ ID NO: 7, and a gene (cscA) encoding the same is preferably represented by a base sequence of SEQ ID NO: 8.

In the present invention, sucrose-6-phosphate hydrolase (SacA) that is one example of the β-fructofuranosidase is an enzyme which is involved in degrading sucrose and linking the degraded products to glycolysis and has an activity to convert sucrose-6-phosphate to glucose-6-phosphate and fructose and an activity to convert sucrose to glucose and fructose. In Examples of the present invention, the β-fructofuranosidase derived from Bacillus subtilis was illustrated, but those derived from other microorganisms also fall within the scope of the present invention and may have an amino acid sequence of SEQ ID NO: 9, and a gene (sacA) encoding the same is preferably represented by a base sequence of SEQ ID NO: 10 (sacA, BSU38040, sucrose-6-phosphate hydrolase).

Moreover, when the amino acid sequence of the Mannheimia-derived β-fructofuranosidase encoded by the sacC gene were compared with the amino acid sequence of enzymes searched through protein BLAST (http://blast.ncbi.nlm.nih.gov/Blast.cgi), invertase encoded by E. coli W-derived cscA has an amino acid sequence identity of 28%, and sucrose-6-phosphate hydrolase (β-fructofuranosidase) encoded by Bacillus subtilis-derived sacA has an amino acid sequence identity of 35%. Also, the β-fructofuranosidases derived from the two strains all have the conserved domain β-fructosidase (COG1621) designated as “SacC”. This indicates that, when any enzyme which has an amino acid sequence somewhat different from the Mannheimia-derived β-fructofuranosidase encoded by the sacC gene, but contains the conserved domain β-fructosidase (COG1621) designated as “SacC”, is introduced into microorganisms as described below, the microorganisms can grow by metabolizing sucrose. Therefore, although the β-fructofuranosidase has been illustrated by an amino acid sequence of SEQ ID NO: 3, 7 or 9, the scope of the present invention is not limited thereto. Namely, all β-fructofuranosidases can be included in the scope of the present invention, as long as they have an activity to hydrolyze the β-D-fructofuranoside bond to liberate fructose. For example, amino acid sequences having an amino acid sequence identity of at least 70%, 80% or 90% to the amino acid sequence of SEQ ID NO: 3, 7 or 9 may also be included in the scope of the present invention.

Likewise, the gene encoding gene the β-fructofuranosidase may have, for example, a base sequence of SEQ ID NO: 4, 8 or 10. DNA comprising a mutation (substitution, deletion, insertion or addition) in one or more bases in these sequences and having a sequence identity of at least 70% or 80%, preferably 90%, and more preferably 95% compared to the base sequence according to the present invention.

The term “sequence identity” as used herein refers to sequence similarity between two polynucleotides or two nucleic acid molecules. The sequence identity can be determined by comparing two optimally aligned sequences over a comparison window, where the fragment of the polynucleotide or amino acid sequence in the comparison window may comprise additions or deletions (e.g., gaps or overhangs) as compared to the reference sequence (e.g., a consensus sequence) for optimal alignment of the two sequences. The percentage of sequence identity can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The sequence identity between nucleic acid or amino acid sequences can be measured using sequence analysis software, for example, BLASTN or BLASTP. The BLAST can generally be used in the website (http://www.ncbi.nlm.nih.gov/BLAST/).

In the present invention, as described above, sucrose which is a disaccharide consisting of D-glucose and D-fructose is known to function to prevent modification of proteins in cells, stabilize proteins in cells and minimize the lysis of cells caused by the change in external environments. Thus, sucrose is highly useful in the production of high concentrations of basic chemical compounds or in high-concentration cell culture.

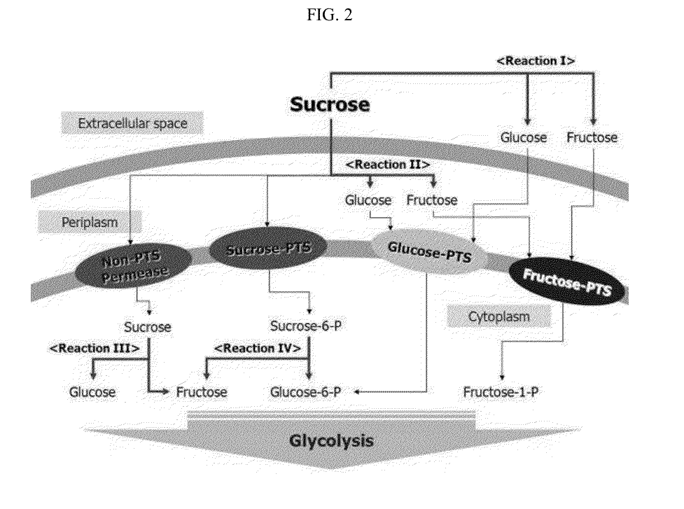

As shown in FIG. 2, when sucrose-6-phosphate hydrolase which is a novel β-fructofuranosidase derived from M. succiniciproducens MBEL55E is introduced into microorganisms incapable of metabolizing sucrose, the introduced enzyme is believed to metabolize sucrose or sucrose-6-phosphate through four possible pathways.

The first possible pathway (Reaction I) is the case in which the sucrose-6-phosphate hydrolase degrades sucrose to glucose and fructose in the extracellular space of cells, and then the degraded products are introduced into cells by enzymes which are involved in transport, such as respective phosphotransferase.

The second possible pathway (Reaction II) is the case in which sucrose-6-phosphate hydrolase degrades sucrose to glucose and fructose in the periplasm, and then the degraded products are introduced into cells by enzymes which are involved in transport, such as respective phosphotransferase.

The third possible pathway (Reaction III) is the case in which sucrose is introduced into cells by permease enzyme other than the phosphotransferase family, and then degraded by the introduced sucrose-6-phosphate hydrolase into glucose and fructose.

The fourth possible pathway (Reaction IV) is the case in which sucrose is converted to sucrose-6-phosphate by phosphate transferase while being introduced into cells, and then converted into fructose and glucose-6-phosphate by the introduced sucrose-6-phosphate hydrolase.

Also, the sacC gene is derived from M. succiniciproducens MBEL55E (KCTC 0769BP) which belongs to the family Pasteurellaceae together with Actinobacillus succinogenes 130Z (ATCC 55618). Particularly, said M. succiniciproducens MBEL55E (KCTC 0769BP) and A. succinogenes 130Z (ATCC 55618) have similar genomic sequences and cell physiologies. The genomic sequences of the two microbial strains are known to be the most similar to each other among all genomic sequences decoded to date (McKinlay et al., Appl. Microbiol. Biotechnol., 76:727, 2007). Also, It is known that the sacC gene derived from A. succinogenes 130Z has a very high homology with one derived from M. succiniciproducens MBEL55E and is the most similar thereto. Thus, it is obvious to those skilled in the art that a M. succiniciproducens MBEL55E-derived sucrose-6-phosphate hydrolase (SacC, MS0909) enzyme and a gene encoding the same, and an A. succinogenes 130Z-derived sucrose-6-phosphate hydrolase (Asuc_{—}1829) enzyme and a gene encoding the same, can likewise be applied in the present invention.

Accordingly, in another still aspect, the present invention relates to a recombinant microorganism capable of metabolizing sucrose in which a gene encoding sucrose phosphotransferase and/or sucrose-6-phosphate hydrolase is introduced, and a method for producing metabolites, biodegradable polymers or recombinant proteins, which comprises culturing said recombinant microorganism in a medium containing sucrose as a carbon source.

In the present invention, the recombinant vector which is a vector capable of expressing a protein in a suitable host cell refers to a DNA construct containing a DNA sequence operably linked to control sequences capable of controlling the expression of a protein together with other sequences which facilitate the manipulation of genes, optimize the expression of proteins or are required for the replication of the vector. The control sequences may include a promoter for regulating transcription, an operator optionally added for regulating transcription, a suitable mRNA ribosome binding site and/or sequences controlling the termination of transcription/translation.

For example, the recombinant vector may be a recombinant vector such as pTrc99A used in Examples of the present invention, but in addition to this vector, other known vectors may be used in the present invention. Also, an expression cassette containing the sacC gene may be inserted not only into expression vectors such as pKK223-3, pTac99A, pET series or pMAL series, but also into a cloning vectors such as pACYC, pBluescript SK-, pBR322, pGEM series or pMB1, and such expression vectors may be used in the present invention. In addition, it is possible to use recombinant vectors known in the art to which the present invention pertains (Sambrook J & Russell D, Molecular Cloning: a laboratory manual, 3rd ed., Cold Spring Harbor Lab (CSHL) Press, New York, 2001). Furthermore, vectors containing, in addition to an ampicillin-resistant gene, several other resistant genes known in the art, may also be used in the present invention.

The above-described genes are derived from M. succiniciproducens MBEL55E (KCTC 0769BP) which belongs to the family Pasteurellaceae together with Actinobacillus succinogenes 130Z (ATCC 55618). Particularly, the two strains, M. succiniciproducens MBEL55E (KCTC 0769BP) and A. succinogenes 130Z (ATCC 55618), have very similar genome sequences and cell physiologies. According to the report (2007) of McKinlay et al., it is known that the genome sequences of the two microbial strains are the most similar to each other among all genome sequences decoded to date (Appl. Microbiol. Biotechnol., 76:727, 2007). Thus, it is obvious to those skilled in the art that the above genes are also applied to genes derived from A. succinogenes 130Z (ATCC 55618) together with M. succiniciproducens MBEL55E (KCTC 0769BP).

In Examples of the present invention, a microorganism incapable of using sucrose as a carbon source was transformed using a recombinant vector containing the ptsG, sacC and rbsK genes, thus constructing a recombinant microorganism having the ability to metabolize sucrose. However, a genome-engineered recombinant microorganism may also be constructed by inserting the above genes into the chromosome of a microorganism incapable of using sucrose as a carbon source according to a method well known in the art.

In the present invention, a recombinant vector containing β-fructofuranosidase-encoding genes including the sacC gene was constructed, and then introduced into E. coli incapable of using sucrose as a carbon source, and the constructed recombinant microorganism was cultured in a medium containing sucrose as a single carbon source. As a result, it was found that the recombinant microorganism had the ability to metabolize sucrose. In still another aspect, the present invention relates to said recombinant vector, a recombinant microorganism transformed with the recombinant vector and capable of metabolizing sucrose, and a method for producing metabolites, biodegradable polymers or recombinant proteins using said recombinant microorganism.

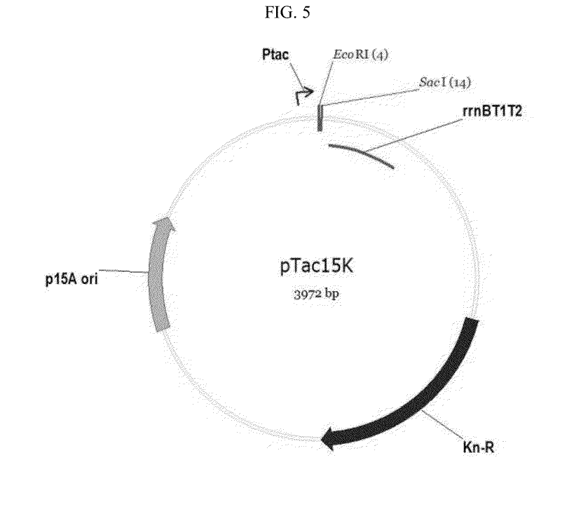

The recombinant vector may be a recombinant vector having a cleavage map of FIG. 5, but in addition to the pTac15K vector shown in FIG. 5, other known vectors may be used in the present invention. Furthermore, an expression cassette containing β-fructofuranosidase-encoding genes including the sacC gene may be inserted not only into expression vectors such as pTrc99A, pTac99A or pMAL series, but also into cloning vectors such as pACYC, pBluescript SK-, pBR322, pGEM series or pMB1, and the recombinant vectors may be applied in the present invention. In addition, it is also possible to use recombinant vectors known in the art to which the present invention pertains (Sambrook J & Russell D, Molecular Cloning: a laboratory manual, 3rd ed., Cold Spring Harbor Lab (CSHL) Press, New York, 2001). Moreover, vectors containing, in addition to a kanamycin-resistant gene, other several resistant genes known in the art, may also be used in the present invention.

In Examples of the present invention, a microorganism incapable of using sucrose as a carbon source was transformed using a recombinant vector containing the sacC, cscA and sacA genes, thus constructing a recombinant microorganism capable of metabolizing sucrose. However, a recombinant microorganism capable of metabolizing sucrose may also be constructed by inserting the above genes into the chromosome of a microorganism incapable of using sucrose as a carbon source according to a method known in the art. In addition, in Examples of the present invention, only a specific E. coli strain was illustrated as a host microorganism incapable of using sucrose as a carbon source, it will be obvious to those skilled in the art that the same result as in the case of using the above E. coli strain can be obtained, even if the above genes are introduced not only into other E. coli strains, but also into host microorganisms incapable of using sucrose as a carbon source, including bacteria, yeasts and fungi.

As used herein, the term “metabolites” refers to a collection of intermediates and products which are produced through metabolic processes. The metabolites are classified into primary metabolites and secondary metabolites. For example, the present invention can be applied in various manners to a recombinant or genome-engineered microorganism incapable of using sucrose in order to produce biofuels, primary and secondary metabolites, biodegradable polymers and recombinant proteins. For the production of, for example, butanol (Atsumi et al., Nature., 451:7174, 2008), ethanol (Lindsay et al., Appl. Microbiol. Biotechnol, 43:70, 1995), and lactic acid (Zhou, Appl. Environ. Microbiol, 69:399 2003), and succinic acid and malic acid (Jantama et al., Biotechnol. Bioeng., 99:1140, 2008), amino acids (Park et al., PNAS, 104:7797, 2006; Lee et al., Molecular Systems Biology, 3:1, 2007), biodegradable polymers (Ahn et al., Appl. Environl. Microbiol., 66:3624, 2000; Park et al., Biomacromolecules, 2:248, 2001; Park et al., Biotechnol. Bioeng., 74:81, 2001), recombinant proteins (Jeong et al., Appl. Environl. Microbiol., 65:3027, 1999; Han et al., Appl. Environl. Microbiol., 69:5772, 2003), glucose has been used as a main carbon source in the prior art to produce the desired bioproducts; however, when the genes of the present invention are introduced into the known microbial strains, the desired bioproducts can be produced using sucrose as a carbon source.

In addition, a person skilled in the art can also easily apply the present invention to produce biodegradable polymers and recombinant proteins. In Examples of the present invention, the production of metabolites such as threonine was illustrated by way of example, but it will be obvious to a person skilled in the art that the present invention can also be easily applied for the production of biodegradable polymers, recombinant proteins and the like.

### EXAMPLES

Hereinafter, the present invention will be described in further detail with reference to examples. It is to be understood, however, that these examples are for illustrative purposes only and are not to be construed to limit the scope of the present invention.

Particularly, in Examples below, a specific E. coli strain was illustrated as a host strain incapable of metabolizing sucrose in order to express the genes of the present invention, but it will be obvious to a person skilled in the art that, even if other E. coli strains or microorganisms of other species are used, metabolites including sucrose can be produced by introducing the genes of the present invention, which are involved in sucrose metabolism, into the microorganisms, and culturing the recombinant microorganisms.

### Example 1

### Examination of the Ability of ptsG, sacC and rbsK Gene to Metabolize Sucrose

1.1: Isolation of ptsG, sacC and rbsK Genes

In order to examine whether genes (ptsG, sacC and rbsK) according to the present invention are involved together in sucrose metabolism, the genes were isolated from M. succiniciproducens MBEL55E (KCTC0769BP).

First, the DNA of ptsG (MS0784) was amplified by PCR using the genomic DNA of M. succiniciproducens MBEL55E (KCTC0769BP) as a template with primers of SEQ ID NOS: 11 and 12. Likewise, the DNAs of sacC (MS0909) and rbsK (MS1233) were amplified by PCR using a set of primers of SEQ ID NOS: 13 and 14 and a set of primers of SEQ ID NOS: 15 and 16, respectively, and overlapping PCR was performed using a mixture of the DNA fragments as a template with primers of SEQ ID NOS: 13 and 16. The ptsG (MS0784), sacC (MS0909) and rbsK (MS1233) are genes encoding sucrose phosphotransferase, sucrose-6-phosphate hydrolase, and fructokinase, respectively.

SEQ ID NO: 11:
5′-GGAATTCATGCTCGTTTTAGCTAGAATTGG
SEQ ID NO: 12:
5′-TCCGAGCTCTTACTATTCTTTTGCGTTAGCTCTTG
SEQ ID NO: 13:
5′-ACCTGCGAGCTCTTTCACACAGGAAACAATTTTCATGCG
GTCGTTTTTACCG
SEQ ID NO: 14:
5′-CAAATTTTGTTTGTCATATGCATGAAATCTGTTTCCTGTG
TGAAATTACTATTTATATTCAATTTCTTTCGGATA
SEQ ID NO: 15:
5′-TATCCGAAAGAAATTGAATATAAATAGTAATTTCACACA
GGAAACAGATTTCATGCATATGACAAACAAAATTTG
SEQ ID NO: 16:
5′-ACCTGCGGGTACCCTATTAGTTTGCTAAAAATTCCGCT

1.2: Construction of Recombinant Vector pMSscrIIA

In order to express the Mannheimia-derived genes, ptsG (MS0784), sacC (MS0909) and rbsK (MS1233), which encode sucrose phosphotransferase, sucrose-6-phosphate hydrolase and fructokinase, respectively, in E. coli, an expression vector was constructed in the following manner.

A DNA fragment containing the sacC (MS0909) and rbsK (MS1233) genes amplified in Example 1.1 was digested with the restriction enzymes SacI and KpnI and ligated into pTrc99A (Pharmacia Biotech., Uppsala, Sweden) digested with the same restriction enzymes, thus constructing pTrc99AsacCrbsK.

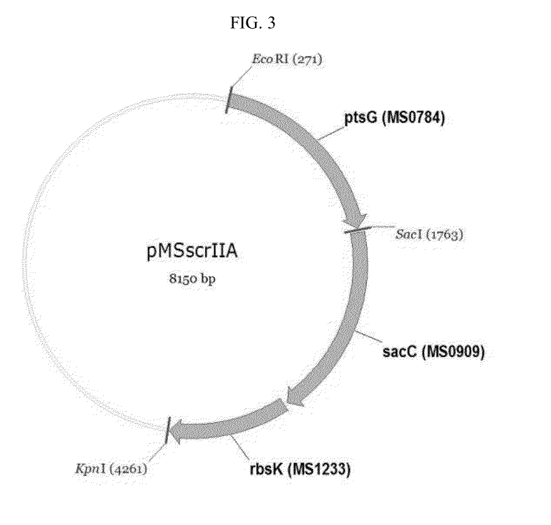

Then, the ptsG (MS0784) DNA fragment obtained in Example 1.1 was digested with EcoRI and SacI and ligated into an expression vector pTrc99AsacCrbsK digested with the same restriction enzymes, thus constructing pTrc99AptsGsacCrbsK. The constructed vector was named “pMSscrIIA” (FIG. 3).

1.3: Construction of Escherichia coli W3110 pMSscrIIA and W3110 pTrc99A Strains

The following experiment was carried out using, as a model microorganism, E. coli W3100 which is a substrain of E. coli K-12 known to be incapable of metabolizing sucrose.

E. coli W3110 was plated on LB solid medium and cultured at 37° C. for 8 hours, and the colony was inoculated into 10 ml of LB liquid medium and cultured for 8 hours. The culture broth was inoculated into 100 mL of LB liquid medium at 1% (v/v) and cultured in a shaking incubator at 37° C.

After about 2 hours, when the culture reached an OD₆₀₀ of about 0.30-0.35, it was left to stand on ice for 20 minutes to stop the growth of the cells. The culture broth was centrifuged at 4° C. at 3,000 rpm for 15 minutes to collect the cells, and then the cells were suspended in 32 ml of RFI solution at 4° C. The suspension was centrifuged at 4° C. at 3,000 rpm for 15 minutes to collect the cells. The cells were re-suspended in 8 ml of RFII solution, and then left to stand on ice for 15 minutes. Finally, the re-suspension was dispensed in an amount of 100 μl/well and stored at −70° C. The composition of RFI solution consisted of 100 mM RbCl, 50 mM MnCl₂-4H₂O, 0.1 M CH₃COOK, 10 mM CaCl₂ and 15% (w/v) glycerol and was adjusted to pH of 5.8 by the addition of 0.2 M acetate. The RFII solution consisted of 10 mM MOPS, 10 mM RbCl, 100 mM CaCl₂ and 15% (w/v) glycerol and was adjusted to pH of 6.8 by the addition of NaOH.

The expression vector pMSscrIIA constructed in Example 1.2 or pTrc99A (Pharmacia Biotech., Uppsala, Sweden) as a control was added to the E. coli W3110 strain, and then the strain was subjected to heat-shock transformation at 42° C. for 90 seconds, thus transforming the strain. After the heat-shock transformation, 0.8 ml of LB liquid medium was added to the strain and the strain was cultured in a shaking incubator at 37° C. for 1 hour.

The culture broth was plated on LB solid medium containing antibiotic ampicillin (final concentration: 50 μg/L) and cultured at 37° C. for 12 hours or more. The formed E. coli W3110 pMSscrIIA and E. coli W3110 pTrc99A colonies were inoculated into LB liquid medium and cultured at 37° C. for 8 hours or more. In order to confirm whether the vector was successfully introduced, the vector was isolated from the cultured strain using GeneAll^{R} Plasmid SV (GeneAll Biotechnology, Korea) and subjected to electrophoresis. E. coli W3110 pMSscrIIA strain was sequenced in Solgent Co. (Korea) using primers of SEQ ID NOS: 17 to 24, thus examining whether the base sequences of the strain were consistent with the base sequences of the genes.

SEQ ID NO: 17:
5′-GGAAACAGACCATGGAATTC
SEQ ID NO: 18:
5′-CCGCAAAAGATTTATTCGAAGAAG
SEQ ID NO: 19:
5′-CCTGGTTATATGATACTTTAGG
SEQ ID NO: 20:
5′-TAGTGCTGGGCGCAAGAGCTAACG
SEQ ID NO: 21:
5′-ACCAGTGGGCGATAAAATCG
SEQ ID NO: 22:
5′-TGATCAAGGTTTCGATTTCT
SEQ ID NO: 23:
5′-TTTTCCTGAATGACGGCGAA
SEQ ID NO: 24:
5′-CGATCTGCCGCAATTTCAAG

1.4: Examination of the Ability of Recombinant E. coli to Metabolize Sucrose

E. coli W3110 pMSscrIIA and E. coli W3110 pTrc99A colonies on solid medium, constructed in Example 1.3, were inoculated into a M9 minimal medium containing 5 g/L of sucrose as a single carbon source and were cultured at 37° C. for 16 hours. Then, the culture broth was inoculated into 100 ml of a M9 minimal medium containing sucrose at 3% (v/v), and then cultured at 37° C. Herein, as an antibiotic, ampicillin was added to a final concentration of 50 μg/L. The M9 minimal medium consisted of 33.9 g/L of Na₂HPO₄, 15 g/L of KH₂PO₄, 2.5 g/L of NaCl, 5 g/L of NH₄Cl and 0.36 g/L of MgSO₄. The concentration of cells in the culture medium was measured as OD₆₀₀ using a spectrophotometer. During the culture period, a sample was periodically collected, the collected sample was centrifuged at 13,000 rpm for 5 minutes, and then the concentration of sucrose in the supernatant was analyzed by high-performance liquid chromatography (HPLC).

As a result, as shown in Table 1, E. coli W3110 pTrc99A strain could not grow in the M9 minimal medium containing sucrose as a single carbon source, but E. coli W3110 pMSscrIIA strain showed an excellent ability to metabolize sucrose. E. coli W3110 pMSscrIIA strain metabolized about 2.2 g/L of sucrose for 19 hours, indicating an increase in biomass of 3.12 based on OD₆₀₀, and produced 0.67 g/L of acetic acid as a byproduct. Thus, it was confirmed that, when a microorganism contains all the ptsG, sacC and rbsK genes, it shows an excellent ability to metabolize sucrose.

TABLE 1

| | | Growth in M9 | | Sucrose | |
|---|---|---|---|---|---|
| | | minimal | Sucrose | concentration | |
| | | medium + | utilizing | (g/L) | |
| Strain | Plasmid | 5 g/L sucrose | phenotype | 0 h | 19 h |
| E. coli W3110 | pTrc99A | − | scr− | 5.23 | 5.22 |
| E. coli W3110 | pMSscrIIA | + | scr+ | 6.73 | 4.53 |

As described above, when either the sucrose phosphotransferase gene or a combination of the phosphotransferase sucrose gene with the sucrose-6-phosphate hydrolase gene was introduced into the microorganism incapable of metabolizing sucrose, the microorganism had the ability to metabolize sucrose and, in addition, produced acetic acid as a metabolite using sucrose as a carbon source.

Accordingly, any person skilled in the art can also easily apply the present invention for the production of, in addition to acetic acid, lactic acid, succinic acid, ethanol, biofuel and bioenergy containing biobutanol, biodegradable polymers and recombinant proteins.

### Example 2

### Examination of the Ability of Each of pstG Gene and sacC Gene to Metabolize Sucrose

2.1: Construction of Recombinant Vector for Examining the Ability of pstG Gene and sacC Gene to Metabolize Sucrose

In order to examine whether the ptsG and sacC genes of the present invention are involved alone in the ability to metabolize sucrose, a vector (pSacHR06ptsG) for deletion of ptsG (MS0784) and a vector (pSacHR06sacC) for deletion of sacC (MS0909) were constructed and subjected to a knock-out experiment.

First, in order to disrupt the sucrose phosphotransferase gene (ptsG) by homologous recombination, a gene exchange vector was constructed in the following manner. The left homologous arm region was amplified using the genomic DNA of Mannheimia succiniciproducens MBEL55E (KCTC0769BP) as a template with primers of SEQ ID NOS: 25 and 26; and the right homologous arm region was amplified using primers of SEQ ID NOS: 27 and 28; a DNA fragment containing an antibiotic marker and a mutant lox site was amplified using a pECmulox vector (Kim et al., FEMS Microbiol. Lett., 278:78, 2008) as a template with primers of SEQ ID NOS: 29 and 30. These three DNA fragments were amplified by overlapping PCR using primers of SEQ ID NOS: 25 and 28.

SEQ ID NO: 25:
5′-ATATCTGCAGCCGGCATTAAATATTAGTCAAC
SEQ ID NO: 26:
5′-CGTTCTAACGGAGGTTGAAAACTGCCCTTT
SEQ ID NO: 27:
5′-GTCTCCCTATCACGCCGTTATTTTCATTATT
SEQ ID NO: 28:
5′-ATTAGTCGACACCATCCCCACGGAATACAT
SEQ ID NO: 29:
5′-TTTCAACCTCCGTTAGAACGCGGCTACAAT
SEQ ID NO: 30:
5′-TAACGGCGTGATAGGGAGACCGGCAGATCC

The final DNA fragment thus amplified was digested with the restriction enzymes PstI and SalI and cloned into pSacHR06 vector (Park et al., PNAS, 104:7797, 2007) digested with the same enzymes, thus constructing pSacHR06ptsG. In addition, pSacHR06sacC was constructed in the same manner as described above using primers of SEQ ID NOS: 31 to 36.

SEQ ID NO: 31:
5′-ATACACTGCAGTTATGCAATTTATCGCACCC
SEQ ID NO: 32:
5′-AATCTGCTCTGATGCGGTCGTGAAATGCTTCCA
SEQ ID NO: 33:
5′-CACAGAATCAGGACAAATGGCATTCAATGCTG
SEQ ID NO: 34:
5′-ATACTGTCGACTCAATGGCATATGCAGCG
SEQ ID NO: 35:
5′-AAGCATTTCACGACCGCATCAGAGCAGATTGTACTGAGAG
SEQ ID NO: 36:
5′-TTGAATGCCATTTGTCCTGATTCTGTGGATAACCGTATTAC

2.2: Construction of M. succiniciproducens MptsG and M. succiniciproducens MsacC Strains

Using each of the exchange vector pSacHR06ptsG for deletion of the ptsG gene and the exchange vector pSacHR06sacC for deletion of the sacC gene, constructed in Example 2.1, the genes were deleted from the genome of M. succiniciproducens MBEL55E (KCTC0769BP) according to the method reported by Kim et al. (FEMS Microbiol. Lett., 278:78, 2008), thus constructing mutant strains, M. succiniciproducens MptsG and M. succiniciproducens MsacC.

Specifically, M. succiniciproducens MBEL55E (KCTC0769BP) was plated on a LB-glucose solid medium containing 10 g/L of glucose and was cultured at 37° C. for 36 hours. Then, the colony was inoculated into 10 ml of LB-glucose liquid medium and cultured for 12 hours. The sufficiently grown cell culture was inoculated into 100 ml of LB-glucose liquid medium at 1% (v/v) and cultured in a shaking incubator at 200 rpm at 37° C.

When the culture broth reached OD₆₀₀ of about 0.3-0.4 after about 4-5 hours, it was centrifuged at 4° C. at 4,500 rpm for 20 minutes to collect the cells, and then the cells were re-suspended in 200 ml of 10% glycerol solution at 4° C. The re-suspension was centrifuged at 4° C. at 5,500 rpm for 20 minutes to collect the cells. The re-suspension process was repeated twice while reducing the glycerol solution to half, and then the cells were re-suspended in glycerol solution at a volume ratio 1:1 to obtain a cell concentrate. The cell concentrate was mixed with each of the gene exchange vectors pSacHR06ptsG or pSacHR06sacC constructed in Example 2.1, and then was subjected to electroporation in the conditions of 2.5 kV, 25 μF and 400 ohms, thus introducing each of the genes into the cultured M. succiniciproducens. After the electroporation, the 1 ml of LB-glucose liquid medium was added to the strain and then the strain was cultured in a shaking incubator at 200 rpm at 37° C. for 1 hour. The culture broth was plated on a LB-glucose solid medium containing antibiotic chloramphenicol (final concentration: 6.8 μg/L) and was cultured at 37° C. for 48 hours or more. In order to select a colony where only double crossover occurred, the formed colonies were streaked on LB-sucrose medium (LB medium containing 100 g/L sucrose) containing chloramphenicol (final concentration: 6.8 μg/L). After 24 hours, the formed colonies were streaked again on the same plate.

The colony (mutant) formed on the plate was cultured in LB-glucose liquid medium containing an antibiotic, and a genomic DNA was isolated from the cultured strain by the method described in Rochelle et al. (FEMS Microbiol. Lett., 100:59, 1992). PCR was performed using the isolated mutant genomic DNA as a template, and the PCR product was electrophoresed to confirm the deletion of ptsG and sacC in each of the mutant strains.

In order to confirm the deletion of ptsG in the MptsG strain, PCRs were performed using a set of primers of SEQ ID NOS: 37 and 38 and a set of primers of SEQ ID NOS: 39 and 40, respectively. In order to confirm the deletion of sacC in the MsacC strain, PCRs were performed using a set of primers of SEQ ID NOS: 39 and and a set of primers of SEQ ID NOS: 41 and 42, respectively.

SEQ ID NO: 37:
5′-CGGGGCGAAAGTGATTGAGA
SEQ ID NO: 38:
5′-AATTGCCGCCTGGGTATTGG
SEQ ID NO: 39:
5′-ACCTTTACTACCGCACTGCTGG
SEQ ID NO: 40:
5′-GCGGGAGTCAGTGAACAGGTAC
SEQ ID NO: 41:
5′-GATCTTGAGTCCGTAAAACAGGCTT
SEQ ID NO: 42:
5′-TTCCGCTCAAGCCATTGTAGTG

### 2.3: Comparison of Growth Between MptsG Strain, MsacC Strain and Parent Strain (MBEL55E)

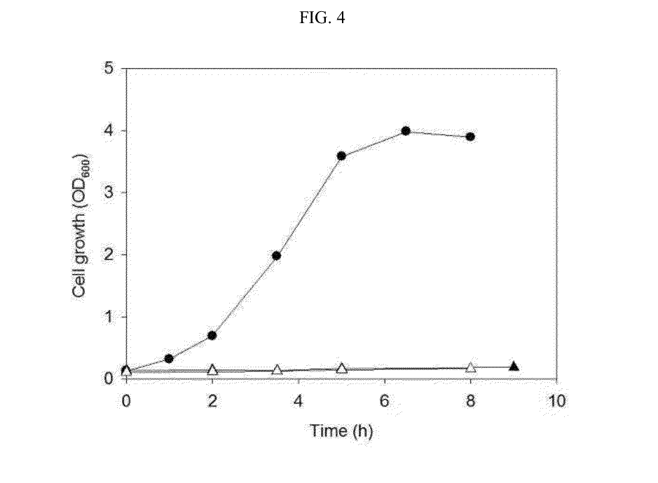

Each of the recombinant MptsG and MsacC strains constructed in Example 2.2 was cultured in BHI (Bacto™ Brain Heart Infusion; Becton, Dickinson and Company, Sparks, Md.) for about 8 hours, and 10 ml of the culture broth was inoculated into 300 ml of MH5S culture medium (per liter: 2.5 g of yeast extract, 2.5 g of polypeptone, 1 g of NaCl, 8.7 g of K₂HPO₄, 10 g of NaHCO₃, 0.02 g of CaCl₂2H₂O, 0.2 g of MgCl₂6H₂O and 10 g of sucrose), and the growth curves of the strains were compared with the growth curve of the parent strain MBEL55E cultured in the same conditions. FIG. 4 is a set of growth curves of the MBEL55E (), MptsG (▴) and MsacC (Δ) strains, measured as OD₆₀₀. As shown in FIG. 4, the growth ability of the parent strain (MBEL55E) in the sucrose medium substantially disappeared when each of the genes was deleted from the strain. This indicates that each of the genes is essential for growth of the strain in the sucrose medium.

Meanwhile, the rbsK gene encoding fructokinase (RbsK, MS1233) was subjected to the same experiment as described above, a change in growth such as a decrease in growth rate was not observed. Also, in measurement results for enzymatic activity, the parent strain and the rbsK-deleted strain showed no great difference in enzymatic activity therebetween and had enzymatic activity levels which were very lower than that of general fructokinase. Namely, the two strains showed negligible enzymatic activities. Based on such results, it is inferred that the rbsK gene does not encode fructokinase or it has a very weak activity, even though it encodes fructokinase.

### 2.4: Comparison of Enzymatic Activities of MptsG and MsacC Strains and Parent Strain

To measure the enzymatic activity of sucrose PTS, the methods of Jacobson et al. (J. Biol. Chem., 254:249, 1979) and Lodge et al. (Infect. Immun., 56:2594, 1988) were used.

First, to permeabilize cells, 1 ml of the cell culture broth at an OD₆₀₀ value of about 1.2 was washed with TDM buffer (50 mM Tris/HCl, 10 mM MgCl₂, 1 mM DTT; pH 7.5) and re-suspended in 1 ml of the same buffer, 0.01 ml toluene was added thereto, and the cell solution was strongly agitated for 45 seconds. The agitated cell solution was centrifuged at 12,000×g, and the collected cells were washed twice with TDM buffer. This procedure was repeated once more, and the resulting cells were re-suspended in 50 μl of TDM buffer, thus preparing permeabilized cells. PEP-dependent sucrose phosphorylation was performed by adding 5 μl of the permeabilized cell suspension to 100 μl of a reaction mixture containing 25 mM Tris/HCl (pH 8.0), 1 mM DTT, 5 mM MgCl₂, 10 mM KF and 1 μCi[U-¹⁴C] sucrose, and measuring the difference in reaction between the mixture containing 1 mM PEP and the mixture not containing PEP. The mixture was allowed to react at 37° C. for 10 minutes, and 1 mL of cold water was added thereto to stop the reaction. The final reaction product was passed through a DEAE filter disk (Whatman, DE81) on a filter system and washed with a 10-fold volume of cold water, and then the radioactivity thereof was measured according to a known method using the Beckman LS6500 liquid scintillation counter (Beckman, Ramsey, Minn.). The activity of sucrose-6-phosphate hydrolase was measured by some modification of the method of Martin et al. (Appl. Environ. Microbiol., 53:2388, 1987). 20 ml of the cell culture at OD₆₀₀ value of about 1 was permeabilized in the same manner as in the measurement of sucrose PTS activity, and finally re-suspended in 1 mL of TDM buffer, thus preparing permeabilized cells. PEP-dependent sucrose phosphorylation was performed by adding 30 μl of the permeabilized cells to 300 μl of a reaction mixture containing 50 mM sodium-potassium phosphate buffer (pH 7.2), 5 mM MgCl₂, 4 mM sucrose, 0.8 mM NADP and 6.4 U glucose-6-phosphate dehydrolase, and measuring the difference in reaction between the mixture containing 10 mM PEP and the mixture not containing PEP.

The activities of the mutant strains from which each of the ptsG and sacC genes has been removed and the activity of the parent strain were measured, and the measurement results are shown in Table 2 below. The results reveal that the sucrose PTS enzyme encoded by MS0784(ptsG) has PTS activity and that the sucrose-6-phosphate hydrolase enzyme encoded by MS0909(sacC) has glycolytic functions in cells.

TABLE 2

| | | | Specific | |
|---|---|---|---|---|
| | | | enzyme | |
| | | Culture | activity | Relative |
| Enzymes | Strains | medium^{a} | (mU/mg)^{b} | activity (%)^{c} |
| Sucrose | MBEL55E | MH5S* | 3.7 | 100.0 |
| phosphotransferase | | BHI | 1.4 | 37.8 |
| | MptsG | BHI | 0.098 | 2.6 |
| Sucrose-6-phosphate | MBEL55E | MH5S* | 18.3 | 100.0 |
| hydrolase | | BHI | 20.4 | 111.5 |
| | MsacC | BHI | 1.7 | 9.3 |

^{a}BHT, BactoTMBrainHeartInfusion (Becton, DickinsonandCompany, Spark, MD); the MH5S medium composition contains: per liter, 2.5 g of yeast extract, 2.5 g of polypeptone, 1 g of NaCl, 8.7 g of K₂HPO₄, 10 g of NaHCO₃, 0.02 g of CaCl₂•H₂O, 0.2 g of MgCl₂•6H₂O and 10 g of sucrose.
^{b}Activity of sucrose phosphotransferase was expressed in a unit of mU/mg converted from cpm (count per minute) measured through a liquid scintillation counter.
^{c}Relative activity was determined by calculating the remaining values for 100% for the culture broth indicated by the symbol *.

### Example 3

### Isolation of Novel Genes Encoding Enzymes Involved in Sucrose Metabolism

Genes encoding β-fructofuranosidase, including sacC, confirmed to have the ability to metabolize sucrose on the basis of the results of Example 2, were isolated from each of Mannheimia, E. coli and Bacillus subtilis in the following manner.

### 3.1: Isolation of Gene Encoding Sucrose-6-Phosphate Hydrolase Derived Form Mannheimia

First, the DNA of the sacC (MS0909) gene was amplified by PCR using the genomic DNA of M. succiniciproducens MBEL55E (KCTC0769BP) as a template with primers of SEQ ID NOS: 43 and 44. The sacC (MS0909) gene is a gene (SEQ ID NO: 4) encoding β-fructofuranosidase (sucrose-6-phosphate hydrolase).

SEQ ID NO 43:
5′-ACTGAGCCATGGCGAAAATCAATAAAGTAGATC-3′
SEQ ID NO 44:
5′-TGATCCGAGCTCCTATTATTCCAGTGTTCCCGCC-3′

3.2: Isolation of Gene Encoding Invertase Derived from E. coli

The DNA of the cscA gene was amplified by PCR using the genomic DNA of E. coli W as a template with primers of SEQ ID NOS: 45 and 46. The cscA is a gene (SEQ ID NO: 8) encoding β-fructofuranosidase (invertase).

SEQ ID NO 45:
ACTCCGGAATTCATGACGCAATCTCGATTGCA
SEQ ID NO 46:
ACCTGCGAGCTCCCGTTGTTCCACCTGATATTATG

3.3: Isolation of Gene Encoding Sucrose-6-Phosphate Hydrolase Derived from Bacillus subtilis

The DNA of the sacA gene was amplified by PCR using the genomic DNA of Bacillus subtilis as a template with primers of SEQ ID NOS: 47 and 48. The sacA is a gene (SEQ ID NO: 10) encoding β-fructofuranosidase (sucrose-6-phosphate hydrolase).

SEQ ID NO 47:
GCATAGAATTCATGACAGCACATGACCAGGAGCT
SEQ ID NO 48:
GCATAGAGCTCCTACATAAGTGTCCAAATTCCGACATTC

### Example 4

### Construction of Recombinant Vectors

4.1: Preparation of pTac15K

A pTac15K vector was constructed by inserting the trc promoter and transcription terminator regions of pKK223-3 (Pharmacia Biotech., Uppsala, Sweden) into pACYC177 (NEB, Beverly, Mass., USA). pTac15K is a expression vector for constitutive expression and has a structure shown in a cleavage map of FIG. 5.

4.2: Construction of pTac15KsacC

In order to express the gene sacC (MS0909) encoding Mannheimia-derived β-fructofuranosidase (sucrose-6-phosphate hydrolase) in E. coli, an expression vector was constructed in the following manner.

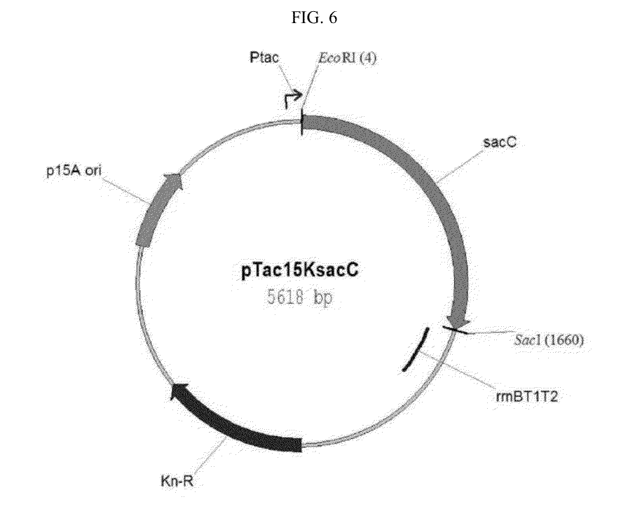

According to a known molecular engineering method, the sacC (MS0909) gene-containing PCR fragment amplified in Example 3.1 was digested with EcoRI and SacI and ligated into pTac15K digested with the same enzymes, thus constructing pTac15KsacC (FIG. 6). The constructed vector was sequenced in Solgent Co. (Korea) using primers of SEQ ID NOS: 49 to 52, thus examining whether the base sequence of the gene introduced into the vector was consistent with that of the sacC (MS0909) gene.

SEQ ID NO 49:
5′-CCCGTTCTGGATAATGTTTT-3′
SEQ ID NO 50:
5′-AAAGTCACGGTTGTTATTCC-3′
SEQ ID NO 51:
5′-CATTTAATGCCGCTCATATT-3′
SEQ ID NO 52:
5′-ACCGCTCAATTATTGAGATT-3′

4.3: Construction of Recombinant Vector pTac15KEWcscA

According to a known molecular engineering method, the DNA fragment obtained in Example 3.2 were digested with EcoRI and SacI and ligated into pTac15K digested with the same enzymes, thus constructing pTac15KEWcscA.

4.4 Construction of Recombinant Vector pTac15 KBSsacA

According to a known molecular engineering method, the DNA fragment obtained in Example 3.3 was digested with EcoRI and SacI and ligated into pTac15K digested with the same enzymes, thus constructing pTac15 KBSsacA.

### Example 5

### Construction of Recombinant Strains

5.1: Construction of Escherichia coli W3110 pTac15KsacC Strain

The following experiment was carried out using, as a model microorganism, E. coli W3110 which is a substrain of E. coli K-12 known to be incapable of metabolizing sucrose. E. coli W3110 strain was plated on LB solid medium and cultured at 37° C. for 8 hours, and then the colony was inoculated into 10 ml of LB liquid medium and cultured for 8 hours. The culture broth was inoculated into 100 ml of LB liquid medium at 1% (v/v) and cultured in a shaking incubator at 37° C.

When the culture broth reached OD₆₀₀ of about 0.30-0.35 after about 2 hours, it was left to stand on ice for 20 minutes to stop the growth of the cells. The culture broth was centrifuged at 4° C. at 3,000 rpm for 15 minutes to collect the cells, and then the cells were suspended in 32 ml of RFI solution at 4° C. The cell suspension was centrifuged at 4° C. at 3,000 rpm for 15 minutes to collect the cells. The collected cells were re-suspended in 8 ml of RFII solution, and then let to stand on ice for 15 minutes. Finally, the re-suspension was dispensed in an amount of 100 μl/well and stored at −70° C. The composition of the RFI solution consisted of 100 mM RbCl, 50 mM MnCl₂-4H₂O, 0.1 M CH₃COOK, 10 mM CaCl₂ and 15% (w/v) glycerol and was adjusted to a pH of 5.8 by the addition of 0.2 M acetate. The RFII solution consisted of 10 mM MOPS, 10 mM RbCl, 100 mM CaCl₂ and 15% (w/v) glycerol and was adjusted to a pH of 6.8 by the addition of NaOH.

The expression vector constructed in Example 2.2 or pTac15K (Pharmacia Biotech., Uppsala, Sweden) as a control was added to E. coli W3110 strain, and then the strain was subjected to heat-shock transformation at 42° C. for 90 seconds, thus transforming the strain. After the heat-shock transformation, 0.8 ml of LB liquid medium was added to the strain and then strain was cultured in a shaking incubator at 37° C. for 1 hour.

The culture broth was plated on a LB solid medium containing antibiotic kanamycin (final concentration: 50 μg/L) and cultured at 37° C. for 12 hours or more. The formed E. coli W3110 pTac15K and E. coli W3110 pTac15KsacC colonies were inoculated into LB liquid medium and cultured at 37° C. for 8 hours or more. In order to confirm whether the vector was successfully introduced into the strain, the vector was isolated from the cultured strain using GeneAll^{R} Plasmid SV (GeneAll Biotechnology, Korea) and subjected to electrophoresis.

5.2: Construction of E. coli W3110 pTac15KEWcscA and E. coli W3110 pTac15 KBSsacA Strains

According to the same method as described in Example 5.1, E. coli W3110 which is a substrain of E. coli K-12 known to be incapable of metabolizing sucrose was transformed using each of the pTac15KEWcscA and pTac15 KBSsacA vectors constructed in Examples 4.3 and 4.4, and whether the vector was successfully introduced was confirmed by electrophoresis.

### Example 6

### Examination of Sucrose-Metabolizing Ability and Growth of Recombinant E. coli

Each of E. coli W3110 pTac15KsacC and E. coli W3110 pTac15K colonies on solid media, constructed in Example 5.1, was inoculated into 10 ml of LB medium and cultured at 37° C. for 8 hours. Then, the cells were inoculated into 100 ml of a M9 minimal medium (containing 10 g/L of sucrose) at 5% (v/v) and cultured at 37° C. Herein, as an antibiotic, kanamycin was added to a final concentration of 50 μg/L. The LB medium consisted of 10 g/L of tryptone, 10 g/L of NaCl and 5 g/L of yeast extract, and the M9 minimal medium consisted of 33.9 g/L of Na₂HPO₄, 15 g/L of KH₂PO₄, 2.5 g/L of NaCl, 5 g/L of NH₄Cl and 0.36 g/L of MgSO₄. The concentration of cells in the culture broth was measured as OD₆₀₀ using a spectrophotometer. During the culture period, a sample was periodically collected, and the collected sample was centrifuged at 13,000 rpm for 5 minutes. Then, the concentrations of sucrose and metabolites in the supernatant were analyzed by high-performance liquid chromatography (HPLC).

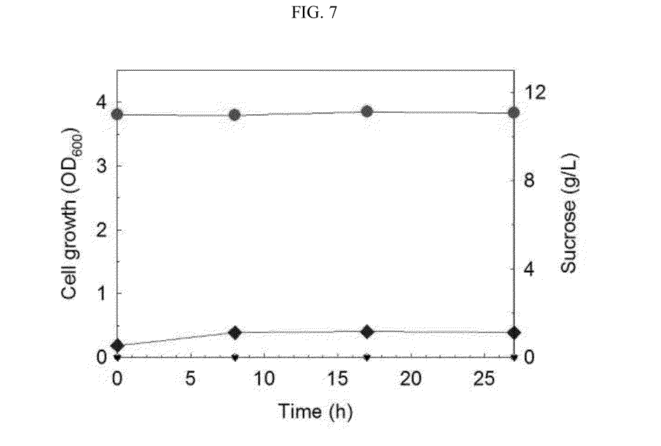

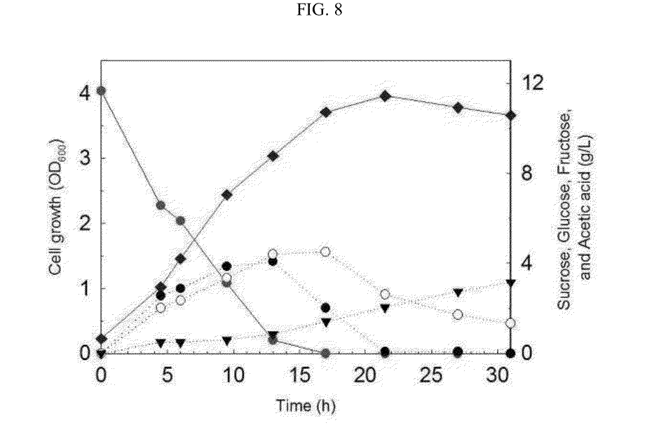

As a result, as shown in FIGS. 7 and 8 and Table 3, E. coli W3110 pTac15K strain could not grow in the M9 minimal medium containing sucrose as a single carbon source, but E. coli W3110 pTac15KsacC strain showed an excellent ability to metabolize sucrose. E. coli W3110 pTac15KsacC strain metabolized 11.08 g/L of sucrose for 17 hours and showed an increase in biomass of 3.71 based on OD₆₀₀. This increase in biomass corresponds to a biomass amount of about 1.37 g/L in view of the conversion factor (1 OD₆₀₀=0.37 g/L DCW) of OD₆₀₀ and dry cell weight (DCW, g/L) of conventional E. coli. In addition, it could be observed that 1.42 g/L of acetic acid was produced, 2.01 g/L of glucose and 4.51 g/L of fructose remained, and the two saccharides were gradually consumed with the passage of time.

TABLE 3

| | | Growth in M9 | Sucrose | Sucrose conc. | |
|---|---|---|---|---|---|
| | | minimal medium + | utilizing | (g/L) | |
| Strains | Plasmids | 10 g/L sucrose | phenotype | 0 h | 17 h |
| E. coli | pTac15K | − | Scr− | 11.00 | 11.12 |
| W3110 | | | | | |
| E. coli | pTac15KsacC | + | Scr+ | 11.08 | 0.00 |
| W3110 | | | | | |

In the growth curve of the W3110 pTac15K strain (FIG. 7), a slight increase in OD at 8 hours after inoculation is believed to be attributable to the components of the medium into which it was inoculated at 5% (v/v). The results of LC analysis of the W3110 pTac15K strain indicated that the stain neither degrade sucrose nor grow using sucrose as a single carbon source.

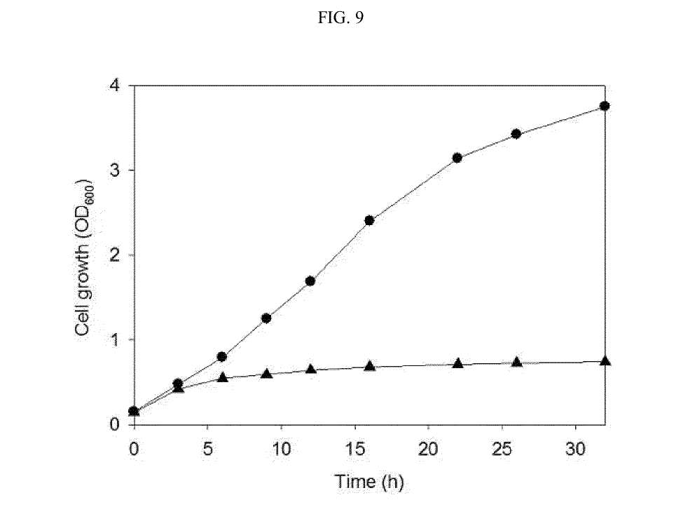

Moreover, the recombinant strains E. coli W3110 pTac15KEWcscA and E. coli W3110 pTac15 KBSsacA transformed in Example 5.2 were cultured using sucrose as a single carbon source in the same manner as in the case of E. coli W3110 pTac15KsacC strain. As a result, as shown in FIG. 9, the strains showed an excellent ability to grow. Also, in the case of E. coli W3110 pTac15KEWcscA, sucrose decreased from 7.77 g/L at the initial stage to 1.82 g/L after 48 hours, and in the case of E. coli W3110 pTac15 KBSsacA strain, sucrose decreased from 8.49 g/L at the initial stage to 7.98 g/L after 48 hours. This suggests that these strains can grow by metabolizing sucrose.

### Example 7

### Production of Metabolites in Recombinant E. coli Using Sucrose as Carbon Source

As described above, when the sucrose-6-phosphate hydrolase derived from M. succiniciproducens was introduced into the microorganism incapable of growing using sucrose as a carbon source, the microorganism could grow using sucrose as a single carbon source. Namely, when a microorganism incapable of using sucrose as a carbon source is treated such that it can cultured in a minimal medium using sucrose as a single carbon source, existing systems for producing various bioproducts (e.g., primary and secondary metabolites, recombinant proteins and biodegradable polymers) can be applied to the present invention.

Accordingly, this Example shows the production of various metabolites using sucrose in anaerobic conditions.

E. coli W3110 pTac15KsacC strain constructed in Example 5.1 was inoculated into 10 ml of LB medium and cultured at 37° C. for 8 hours, and then the culture broth was inoculated into 200 mL of LB medium and cultured at 37° C. for 8 hours. Then, the culture broth was inoculated into a 2.5-L volume fermentor (New Brunswick System, BioFlo 3000). A culture medium in the fermentor consisted of an R/2 minimal medium containing 20 g/L of sucrose, and 100% CO₂ was introduced into the fermentor at a rate of 0.5 vvm under operating conditions of pH 6.8, 37° C. and 200 rpm. As an antibiotic, kanamycin was added to a final concentration of 50 μg/L. The R/2 medium consisted of 6.75 g/L of KH₂PO₄, 2 g/L of (NH₄)₂HPO₄, 0.85 g/L of C₆H₈O₇H₂O, 0.7 g/L of MgSO₄7H₂O and 5 ml/L of trace metal solution (10 g/L FeSO₄7H₂O, 2 g/L CaCl₂, 2.2 g/L ZnSO₄7H₂O, 0.54 g/L MnSO₄5H₂O, 1 g/L CuSO₄5H₂O, 0.1 g/L NH₄Mo₇O₂₄7H₂O, 0.02 g/L Na₂B₄O₇10H₂O, and 5 mL HCl). The concentration of cells in the culture broth was measured as OD₆₀₀ using a spectrophotometer, and during the culture period, a sample was periodically collected, the collected sample was centrifuged at 13,000 rpm for 5 minutes, and then the concentrations of sucrose and metabolites in the supernatant were analyzed by high-performance liquid chromatography (HPLC).

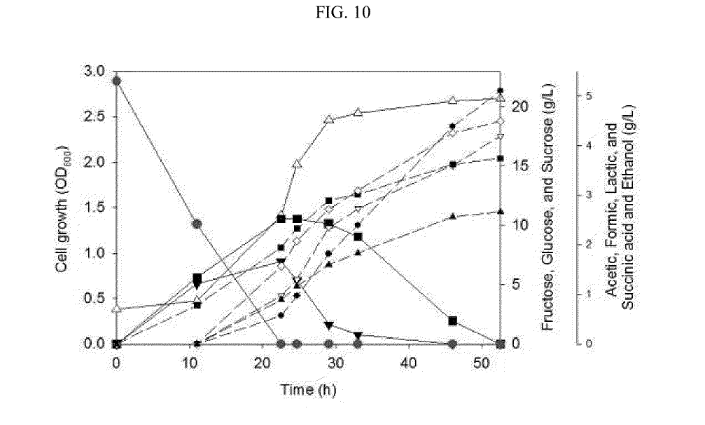

As a result, as shown in FIG. 10 and Table 4, acetic acid, formic acid, lactic acid, succinic acid and ethanol could be successfully produced. In the above-described anaerobic conditions for 52.5 hours, 22.13 g/L of sucrose and saccharides derived therefrom were completely consumed and, as a result, 4.49 g/L of acetic acid, 3.74 g/L of formic acid, 4.19 g/L of lactic acid, 5.10 g/L of succinic acid and 2.66 g/L of ethanol were produced at yields of 0.20, 0.17, 0.19, 0.23, and 0.12 g/g of sucrose, and the total sum of these yields was 0.91 g/g of sucrose which significantly approached the theoretical value. Also, OD₆₀₀ at 52.5 hours after the start of the culture was 2.7 which corresponded to a g DCW of 0.999 in view of the above-described conversion factor. The calculated specific yield indicating yield per strain weight is shown in Table 4 below. The results of Table 4 indicate that, when the sucrose-6-phosphate hydrolase is introduced alone, various metabolites can be successfully produced at high yield and high specific yield using sucrose.

TABLE 4

| Sucrose and | | | Yield | Specific yield |
|---|---|---|---|---|
| products | Initial conc. | Final conc. | (g/g sucrose) | (g/g DCW) |
| Sucrose | 22.13 | 0 | − | − |
| Acetic acid | 0 | 4.49 | 0.20 | 4.49 |
| Formic acid | 0 | 3.74 | 0.17 | 3.74 |
| Lactic acid | 0 | 4.19 | 0.19 | 4.19 |
| Succinic acid | 0 | 5.10 | 0.23 | 5.11 |
| Ethanol | 0 | 2.66 | 0.12 | 2.66 |
| Total | 22.13 | 20.18 | 0.91 | 20.19 |

### Example 8

### Application to Metabolically Engineered Useful Bioproducts Through Application to Threonine-Producing Strain

The following experiment was carried out using a threonine-producing strain in order to illustrate that when the genes of the present invention are introduced into a genome-engineered or recombinant E. coli strain, the strain can also produce bioproducts using sucrose instead of other existing carbon sources. Specifically, the following experiment was carried out using, as a model, a genome-engineered and recombinant TH28C pBRThrABCR3 (Lee et al., Molecular Systems Biology, 3:149, 2007) metabolically modified based on E. coli W3110.

According to the same method as described in Example 5, the TH28C pBRThrABCR3 strain was transformed with the plasmid pTac15KsacC constructed in Example 4, thus constructing TH28C pBRThrABCR3 pTac15KsacC.

The constructed TH28C pBRThrABCR3 pTac15KsacC strain was inoculated into mL of LB medium and cultured at 31° C. for 12 hours, and the culture broth was inoculated into 50 mL of LB medium and cultured at 31° C. for 12 hours. Then, the culture broth was inoculated into a 2.5-L volume fermentor (New Brunswick System, BioFlo 3000). The culture medium contained in the fermentor consisted of a TPM2 medium containing 20 g/L of sucrose and was maintained at an air saturation level of more than 40% by supplying air having a dissolved oxygen concentration of 1 vvm under conditions of pH of 6.5 and temperature of 31° C. and automatically increasing revolution speed (rpm) to 1000. As antibiotics, chloramphenicol, kanamycin and ampicillin were added to final concentrations of 30 μg/L, 40 μg/L and 50 μg/L, respectively. The TPM2 medium consisted of 2 g/L of yeast extract, 2 g/L of MgSO₄.7H₂O, 2 g/L of KH₂PO₄, 10 g/L of (NH₄)₂SO₄, 0.2 g/L of L-methionine, 0.2 g/L of L-lysine, 0.05 g/L of L-isoleucine, and 10 ml/L of trace metal solution (10 g/L FeSO₄7H₂O, 2 g/L CaCl₂, 2.2 g/L ZnSO₄7H₂O, 0.54 g/L MnSO₄5H₂O, 1 g/L CuSO₄5H₂O, 0.1 g/L NH₄Mo₇O₂₄7H₂O, 0.02 g/L Na₂B₄O₇10H₂O, and 5 mL HCl). The concentration of cells in the culture broth was measured as OD₆₀₀ using a spectrophotometer, and the periodically collected sample was centrifuged at 13,000 rpm for 5 minutes, and then the concentrations of sucrose and metabolites in the supernatant was analyzed by high-performance liquid chromatography (HPLC). For analysis of amino acids, 5 ml of the culture broth was subjected to centrifugation and filtration processes, and then separated in Science Lab Center Co. (Daejeon, Korea) using a cation separation column (LCA K06/Na 1.6150 mm; SykamGmbH, Eresing, Germany). Then, the separated sample was analyzed using the Sykam S433 amino-acid analyzer.

As a result, as shown in Table 5 below, 4.7 g/L of threonine could be successfully produced using sucrose.

TABLE 5

| | | Final value |
|---|---|---|
| | Initial value | (after 15 h culture) |
| OD₆₀₀ | 0.4 | 18.0 |
| Sucrose | 23.7 g/L | 0 g/L |
| Threonine | 0 g/L | 4.7 g/L |

As described above, when the β-fructofuranosidase gene of the present invention was introduced into a microorganism incapable of using sucrose as a carbon source, the microorganism had have the ability to metabolize sucrose and, in addition, could produce various metabolites, including acetic acid, formic acid, lactic acid, succinic acid and ethanol, using sucrose as a carbon source. Moreover, when the gene of the present invention was introduced into the metabolically modified strain in the same manner, a desired metabolite (threonine in this Example) could be successfully produced. In addition, any person skilled in the art can also easily apply the present invention to produce biodegradable polymers, recombinant proteins and the like.

### INDUSTRIAL APPLICABILITY

As described above in detail, the present invention provides a recombinant microorganism capable of using inexpensive sucrose as a carbon source instead of expensive glucose. Also, in a process of culturing microorganisms which have been incapable of using sucrose as a carbon source, sucrose can substitute for other carbon sources including glucose. In the present invention, a group of novel enzymes enabling the efficient use of sucrose which is inexpensive and abundant in nature was identified and developed. Such enzymes will be used for the more efficient and economical production of useful chemical compounds or medical recombinant proteins through microbial fermentation using sucrose as a carbon source. Particularly, because sucrose is known to function to prevent modification of proteins in cells, stabilize proteins in cells and minimize the lysis of cells, it is highly useful in the production of high concentrations of basic chemical compounds or in high-concentration cell culture.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1.-22. (canceled)

23. A recombinant vector containing a gene (ptsG) encoding a sucrose phosphotransferase and a sacC gene encoding sucrose-6-phosphate hydrolase, wherein the ptsG gene is represented by a base sequence of SEQ ID NO:2.

24. The recombinant vector according to claim 23, wherein the sacC gene is represented by a base sequence of SEQ ID NO: 4.

25. A recombinant microorganism capable of metabolizing sucrose in which the recombinant vector of claim 23 is introduced into a host cell selected form the group consisting of bacteria, yeast and fungi.

26. A recombinant microorganism capable of metabolizing sucrose in which a gene (ptsG) encoding a sucrose phosphotransferase and a gene (sacC) encoding sucrose-6-phosphate hydrolase is introduced into a chromosomal DNA of a host cell selected form the group consisting of bacteria, yeast and fungi, wherein the ptsG gene is represented by a base sequence of SEQ ID NO:2.

27. The recombinant microorganism capable of metabolizing sucrose according to claim 26, wherein the sacC gene is represented by a base sequence of SEQ ID NO: 4.

28. A recombinant microorganism capable of metabolizing sucrose in which a gene encoding a β-fructofuranosidase having an activity to hydrolyze β-D-fructofuranoside bond to liberate fructose is introduced into a chromosomal DNA of a host cell selected form the group consisting of bacteria, yeast and fungi.

29. A method for producing metabolites, biodegradable polymers or recombinant proteins, the method comprises;
(a) preparing a recombinant microorganism capable of metabolizing sucrose in which the gene represented by SEQ ID NO:4 is introduced into a chromosomal DNA of a host cell selected from the group consisting of bacteria and fungi,
(b) culturing the recombinant microorganism in a medium containing sucrose as a carbon source.

30. The method according to claim 29, wherein the recombinant microorganism capable of metabolizing sucrose is E. coli.


SEQUENCE LISTING (52 sequences)

SEQ ID NO: 1 - protein, 492 aa, Artificial Sequence
  Synthetic Construct
MLVLARIGENFCLIYKRGVAMNYPKIAQQVIEKLGGKENIANLAHCATRLRLTMNDESKIDKQAIEDIEGVKGQFSTSGQYQIIFGSGTVNKVYAEMNTIMNGSPSADSTGESQQAKGPQQGLIQRLIKGLADIFVPIIPAIVAGGLLMGINNVFTAKDLFEEGRTLLDLYPQYKDLADLINTFANAPFVFLPVLLGFSATRKFGGNPFLGATLGMLLVHPALTNAYGYAEALAGGNLQLWNIFGLEIEKVGYQGTVIPVLIAAWVLATLEKFLVKVVPSVLNNLVTPLFSLFITGFLAFTVIGPFGREAGEFLSQGLTWLYDTLGFIGGGVFGALYAPIVITGMHQTFIAIETQLLASTAATFIFPIAAMSNIAQGAACLAVAVLNKDAKTRGLALPSGISALLGITEPAMFGVNLRFRYPFYAAMLGAGSAAAFIAFFNVKATALGAAGLIGIASIRAGDWGMYSVGMVISFCVAFAAALVLGARANAKE

SEQ ID NO: 2 - DNA, 1479 bp, Artificial Sequence
  Synthetic Construct
ttgctcgttttagctagaattggcgaaaatttttgcttaatttataaacgaggagtcgctatgaactaccctaaaattgcccaacaggttatcgaaaaacttggcggaaaagaaaatatcgctaatcttgcgcattgtgcaacgcgtttgcgcttgacaatgaatgacgaaagtaaaatcgacaaacaggccattgaagatatcgagggcgtaaaagggcagttttcaacctccggtcaataccaaattattttcggttcaggtacggtgaataaagtttacgccgaaatgaataccattatgaacggttcgccgtcggcggattccaccggggaaagtcaacaggcgaaagggccgcagcaaggtttgattcaacgattaattaaaggtctggctgatattttcgttcccattattccggctattgtcgccggcggtttgttaatggggattaataatgtctttaccgcaaaagatttattcgaagaagggaggacattactcgacctttatccgcaatacaaagatttagcggatttaattaatacctttgctaacgcgccttttgtgtttctgcccgtattgttaggtttctcggcaaccagaaaattcggtggcaatccgttcttaggagcgacattaggtatgttgctcgttcaccccgctttaaccaatgcttacggttatgcggaagcgttagccggcggcaatcttcaattatggaatattttcgggctagagattgaaaaagtcggttatcaaggtacggttattcccgttttaattgccgcctgggtattggcgactttagaaaaattcttagtgaaagtagtgccttccgtattaaataatttagtcacgccgttattttcattatttatcaccggttttttggctttcaccgtaatcggacctttcggtcgtgaagcgggggaatttttaagtcagggtttaacctggttatatgatactttaggttttatcggcggcggcgtgttcggcgcattatacgcacctatcgtgattaccggtatgcaccaaacctttatcgccattgaaacgcaattgctggcaagcactgcggcaacttttatcttcccgattgccgccatgtcgaatattgcgcagggtgccgcttgtttagccgttgccgtgttaaataaagatgccaaaacccgaggtctggcgttgccttccggtatttccgcattattagggattaccgaacctgccatgttcggggtgaatttgcgcttccgttatccgttctatgcggctatgttaggtgccggttccgccgcggcgtttatcgcgttcttcaatgttaaagccactgcgcttggcgcggcgggcttaatcggtatcgcatcaattcgtgccggcgactggggaatgtattccgtggggatggtaatttcgttttgtgtggctttcgctgcggcattagtgctgggcgcaagagctaacgcaaaagaatag

SEQ ID NO: 3 - protein, 502 aa, Artificial Sequence
  Synthetic Construct
MRSFLPHFSLFYFHQGIMMIIFNNGKYKSILAAEQGELERIKSEVEKDRDFRPYYHLAPSTGLLNDPNGLVFDGEKFHLFYQWFPFDAIHGMKHWKHFTTEDFHIYTEADPLIPCELFESHGCYSGGALPVGDKIAAFYTGNTRRAADNQRVPFQNLAIFDRTGKLLSKRPLIENAPKGYTEHVRDPKPYFTKEGKIRFICGAQREDLTGTAIIFEMDNLDDEPRLLGELSLPAFDNQKVFMWECPDLLKVGDNDIFIWSPQGKRREARRFQNNFHAVYAVGKLDDRTFNAAHIAELDQGFDFYAPQTFAGLENQKHAVMFGWCGMPDLTYPTDKYKWHSMLTLPREITLQGNRLVQRPIKEIYQNLTALSQISLQQQAEIQDLDRAYIKFDAENTAFNIRFFANEQGQTLSLSYDGELVCLDRSQTEETEWMKKFASQRYCEIKNLRQVEIFFDRSIIEIFLNDGEKALTSRFFIANRQNSVKTDRTLRLNVGYPKEIEYK

SEQ ID NO: 4 - DNA, 1509 bp, Artificial Sequence
  Synthetic Construct
gtgcggtcgtttttaccgcacttttctttattttattttcaccaaggtataatgatgattatatttaataacggtaaatataaaagcattttggcggccgaacagggcgagcttgaacgaattaaaagcgaggtagaaaaagatcgggattttcgcccctactaccatctcgcgccatctacaggcttactaaacgatcccaacggtttggtttttgacggcgaaaaatttcatctgttctatcaatggttcccgtttgatgccattcacggcatgaaacactggaagcatttcacgaccgaggattttcatatctataccgaagccgatccgcttatcccttgcgaactttttgaaagtcacggttgttattccggcggcgccttaccagtgggcgataaaatcgccgcattttataccggtaacacaagacgcgctgcggataaccaacgggttccctttcaaaatttagcgatttttgaccgcaccggtaaacttctcagtaaacgcccattaattgaaaatgcaccgaaaggctacaccgaacacgttcgtgatccgaaaccttacttcacaaaagaaggaaaaatccgttttatttgcggcgcacaacgtgaagatttaaccggcaccgccattatttttgaaatggataatcttgatgatgagccgcgcttattaggcgaattgtctctccccgcttttgataatcaaaaggtgtttatgtgggaatgcccggatttattgaaagtcggcgataacgatattttcatctggtctccgcaaggcaaacggcgcgaagcccgccggttccaaaataattttcatgcggtctatgccgtaggaaaattggatgatcggacatttaatgccgctcatattgccgaacttgatcaaggtttcgatttctatgcgccgcaaacttttgccggcctggaaaatcaaaagcatgccgttatgttcggttggtgcggtatgccggatttgacctacccgacggataaatacaaatggcattcaatgctgactctcccgcgggaaattacattgcaaggcaataggcttgttcagcgcccgataaaagaaatttatcaaaatttgaccgcactttcgcaaatttccctgcaacagcaagcggaaattcaggatttagatcgagcctatattaaatttgacgcggaaaacacagcgtttaatatccgcttttttgccaacgaacaaggacaaacgctctcgctttcttatgacggagaacttgtttgtctggatcgttcgcaaacggaagaaacggaatggatgaaaaaatttgctagtcagcgttattgtgaaataaagaatctgcgacaagtggaaattttctttgaccgctcaattattgagattttcctgaatgacggcgaaaaagccctgacttcgagattctttattgcgaaccgccaaaattccgtcaaaaccgaccgcactttgcggttaaacgtcggttatccgaaagaaattgaatataaatag

SEQ ID NO: 5 - protein, 310 aa, Artificial Sequence
  Synthetic Construct
MHMTNKIWVLGDAVVDLIPDGDNHYLRCAGGAPANVAVGVARLGVPSAFIGRVGKDPLGEFMRDTLNQENVNTDYMLLDPKQRTSTVVVGLTDGERSFTFMVNPSADQFLQISDLPQFQAGDWLHCCSIALINEPTRSATFTAMKNIRAAGGKVSFDPNLRESLWKSQDEMIDVVMEAVSLADVLKFSEEELTLLTHTDSLEKSFEKITALYPDKLIIVTLGKEGALYHLHGKKEVVAGKALKPVDTTGAGDAFVSGLLAGLSQTENWQQPEQLVTIIRQANASGALATTAKGAMSALPNQQQLAEFLAN

SEQ ID NO: 6 - DNA, 933 bp, Artificial Sequence
  Synthetic Construct
atgcatatgacaaacaaaatttgggtattaggcgatgccgtggtggatttaattcctgacggagacaaccattatttgcgttgcgcaggcggcgcaccggctaatgtggcggtcggcgttgcccgtttaggtgtgcctagcgcatttatcggccgtgtaggtaaagatccgttaggggaatttatgcgcgatacgctgaatcaggaaaatgtaaacaccgattatatgttgttagatcctaaacaacgtacttcgacggtggtggttggattaactgacggcgaacgtagttttacctttatggtgaatccaagtgcggatcaatttttacaaatttccgatctgccgcaatttcaagccggagactggttgcactgctgctctatcgccttaatcaatgaaccgacccgcagcgctactttcacggcaatgaaaaatatccgtgcggccggcggtaaagtatctttcgatccgaatttacgcgaaagcttatggaaatcccaggatgaaatgatcgatgtggtgatggaagcggtaagccttgccgacgtattgaaattttcagaagaagaattaacgctgttaacccataccgacagcctggaaaaatcttttgaaaaaatcaccgcactttatcccgataaattgattattgtcactttagggaaagaaggtgcgctctatcatctgcacggtaaaaaagaggtggttgcagggaaagcgctgaaaccggtagataccaccggggccggcgacgcttttgtcagcgggttattagccggattatcacaaacggaaaactggcagcaacctgaacaactcgttactattattcgccaggccaacgccagcggcgcgcttgccacaacggcaaaaggcgctatgtcggcattaccgaatcagcaacaattagcggaatttttagcaaactaa

SEQ ID NO: 7 - protein, 477 aa, Artificial Sequence
  Synthetic Construct
MTQSRLHAAQNALAKLHEHRGNTFYPHFHLAPPAGWMNDPNGLIWFNDRYHAFYQHHPMSEHWGPMHWGHATSDDMIHWQHEPIALAPGDDNDKDGCFSGSAVDDNGVLSLIYTGHVWLDGAGNDDAIREVQCLATSRDGIHFEKQGVILTPPEGIMHFRDPKVWREADTWWMVVGAKDPGNTGQILLYRGSSLREWTFDRVLAHADAGESYMWECPDFFSLGDQHYLMFSPQGMNAEGYSYRNRFQSGVIPGMWSPGRLFAQSGHFTELDNGHDFYAPQSFLAKDGRRIVIGWMDMWESPMPSKREGWAGCMTLARELSESNGKLLQRPVHEAESLRQQHQSVSPRTISNKYVLQENAQAVEIQLQWALKNSDAEHYGLQLGTGMRLYIDNQSERLVLWRYYPHENLDGYRSIPLPQRDTLALRIFIDTSSVEVFINDGEAVMSSRIYPQPEERELSLYASHGVAVLQHGALWLLG

SEQ ID NO: 8 - DNA, 1434 bp, Artificial Sequence
  Synthetic Construct
atgacgcaatctcgattgcatgcggcgcaaaacgccctagcaaaacttcatgagcaccggggtaacactttctatccccattttcacctcgcgcctcctgccgggtggatgaacgatccaaacggcctgatctggtttaacgatcgttatcacgcgttttatcaacatcatccgatgagcgaacactgggggccaatgcactggggacatgccaccagcgacgatatgatccactggcagcatgagcctattgcgctagcgccaggagacgataatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagatccaggcaacacggggcagatcctgctttatcgcggcagttcgttgcgtgaatggaccttcgatcgcgtactggcccacgctgatgcgggtgaaagctatatgtgggaatgtccggactttttcagccttggcgatcagcattatctgatgttttccccgcagggaatgaatgccgagggatacagttaccgaaatcgctttcaaagtggcgtaatacccggaatgtggtcgccaggacgactttttgcacaatccgggcattttactgaacttgataacgggcatgacttttatgcaccacaaagctttttagcgaaggatggtcggcgtattgttatcggctggatggatatgtgggaatcgccaatgccctcaaaacgtgaaggatgggcaggctgcatgacgctggcgcgcgagctatcagagagcaatggcaaacttctacaacgcccggtacacgaagctgagtcgttacgccagcagcatcaatctgtctctccccgcacaatcagcaataaatatgttttgcaggaaaacgcgcaagcagttgagattcagttgcagtgggcgctgaagaacagtgatgccgaacattacggattacagctcggcactggaatgcggctgtatattgataaccaatctgagcgacttgttttgtggcggtattacccacacgagaatttagacggctaccgtagtattcccctcccgcagcgtgacacgctcgccctaaggatatttatcgatacatcatccgtggaagtatttattaacgacggggaagcggtgatgagtagtcgaatctatccgcagccagaagaacgggaactgtcgctttatgcctcccacggagtggctgtgctgcaacatggagcactctggctactgggttaa

SEQ ID NO: 9 - protein, 480 aa, Artificial Sequence
  Synthetic Construct
MTAHDQELRRRAYEEVEKKEPIANSDPHRQHFHIMPPVGLLNDPNGVIYWKGSYHVFFQWQPFQTGHGAKFWGHYTTQDVVNWKREEIALAPSDWFDKNGCYSGSAVTKDDRLYLFYTGNVRDQDGNRETYQCLAVSDDGLSFEKKGVVARLPEAILTAHFSRSEVWEHEGTWYMVIGAQTENLKGQAVLFASDNLTEWRFLGPITGAGFNGLDDFGYMWECPDLFSLQGSDVLIVSPQGLEADGFRYQNVYQSGYFVGRLDYNKPELKHGEFTELDQGFDFYAPQTLEDDQGRRILFAWMAVPDQDEGSHPTIDCHWIHCMTLPRQLTLSGQKLIQQPLPELKAMRRNEKKIHINMHGSSGALPVEKPERTEILLEDIHTESGFSISIRGTATFSFHKDEGIVTLERKSFDGKRTEARHCRIKDLHTVHMFLDASSVEIFINNGEEVFSARYFPFPGNHEVTASATGKSEMNVGIWTLM

SEQ ID NO: 10 - DNA, 1443 bp, Artificial Sequence
  Synthetic Construct
atgacagcacatgaccaggagcttcgtcgccgggcttatgaagaagtggagaaaaaagagcccatcgctaacagcgatccgcaccgccagcattttcatatcatgccgccggttgggctgctgaatgacccgaatggcgtgatttattggaagggcagctatcatgtattctttcagtggcagccgtttcagacggggcacggcgcaaaattttgggggcattatacgacacaggatgttgtgaattggaagcgggaagagattgcgctggctccgagtgattggtttgataaaaacggctgctactcgggcagcgctgtcacgaaagacgatcggctctatcttttttacacaggaaatgtcagggatcaggatggaaatcgggaaacgtatcaatgccttgctgtttctgacgacgggctgtcctttgagaaaaagggtgtcgtcgcccgccttccggaagcgatattaacggcgcacttttcgcgatccgaagtatgggagcatgaaggcacatggtatatggtgattggtgcgcaaacagagaatttgaaagggcaggctgtgttgtttgcttctgataacctgacagagtggagatttcttggcccgataaccggcgcgggcttcaacgggctggacgattttggatacatgtgggaatgccctgatttgttttcccttcaaggatcggatgtgctgattgtttcgcctcaagggcttgaggctgacggtttccgttatcagaacgtatatcaatcaggttattttgtcggccgcctcgattataacaagcctgaactgaagcatggtgaatttacggagcttgatcaaggttttgatttttacgcgccgcaaacacttgaagacgatcagggaaggcggattttatttgcatggatggcggtgcctgatcaggatgaagggtcccatccgaccattgactgccactggattcactgcatgacgctgccgagacagctgacgctttcaggacagaagctgattcagcagccgctgcctgagctaaaagccatgcgcagaaatgagaaaaaaatacacatcaacatgcatggatcatctggtgcgcttccagtggaaaaacctgaaagaactgagattctactggaagacattcatacggagtctggcttttcaatcagtatccgcggaacggctacgttttccttccataaagacgaggggattgttacgctggaacgaaagagctttgacggaaaaagaacagaagcgagacattgccgcatcaaggatttgcataccgtacacatgtttctcgacgcgtcatctgtggaaatctttatcaataacggagaagaggtctttagtgcaagatattttcctttcccgggaaatcatgaagtaacagccagtgcgaccgggaaatctgaaatgaatgtcggaatttggacacttatgtag

SEQ ID NO: 11 - DNA, 30 bp, Artificial Sequence
  Synthetic Construct
ggaattcatgctcgttttagctagaattgg

SEQ ID NO: 12 - DNA, 35 bp, Artificial Sequence
  Synthetic Construct
tccgagctcttactattcttttgcgttagctcttg

SEQ ID NO: 13 - DNA, 52 bp, Artificial Sequence
  Synthetic Construct
acctgcgagctctttcacacaggaaacaattttcatgcggtcgtttttaccg

SEQ ID NO: 14 - DNA, 75 bp, Artificial Sequence
  Synthetic Construct
caaattttgtttgtcatatgcatgaaatctgtttcctgtgtgaaattactatttatattcaatttctttcggata

SEQ ID NO: 15 - DNA, 75 bp, Artificial Sequence
  Synthetic Construct
tatccgaaagaaattgaatataaatagtaatttcacacaggaaacagatttcatgcatatgacaaacaaaatttg

SEQ ID NO: 16 - DNA, 38 bp, Artificial Sequence
  Synthetic Construct
acctgcgggtaccctattagtttgctaaaaattccgct

SEQ ID NO: 17 - DNA, 20 bp, Artificial Sequence
  Synthetic Construct
ggaaacagaccatggaattc

SEQ ID NO: 18 - DNA, 24 bp, Artificial Sequence
  Synthetic Construct
ccgcaaaagatttattcgaagaag

SEQ ID NO: 19 - DNA, 22 bp, Artificial Sequence
  Synthetic Construct
cctggttatatgatactttagg

SEQ ID NO: 20 - DNA, 24 bp, Artificial Sequence
  Synthetic Construct
tagtgctgggcgcaagagctaacg

SEQ ID NO: 21 - DNA, 20 bp, Artificial Sequence
  Synthetic Construct
accagtgggcgataaaatcg

SEQ ID NO: 22 - DNA, 20 bp, Artificial Sequence
  Synthetic Construct
tgatcaaggtttcgatttct

SEQ ID NO: 23 - DNA, 20 bp, Artificial Sequence
  Synthetic Construct
ttttcctgaatgacggcgaa

SEQ ID NO: 24 - DNA, 20 bp, Artificial Sequence
  Synthetic Construct
cgatctgccgcaatttcaag

SEQ ID NO: 25 - DNA, 32 bp, Artificial Sequence
  Synthetic Construct
atatctgcagccggcattaaatattagtcaac

SEQ ID NO: 26 - DNA, 30 bp, Artificial Sequence
  Synthetic Construct
cgttctaacggaggttgaaaactgcccttt

SEQ ID NO: 27 - DNA, 31 bp, Artificial Sequence
  Synthetic Construct
gtctccctatcacgccgttattttcattatt

SEQ ID NO: 28 - DNA, 30 bp, Artificial Sequence
  Synthetic Construct
attagtcgacaccatccccacggaatacat

SEQ ID NO: 29 - DNA, 30 bp, Artificial Sequence
  Synthetic Construct
tttcaacctccgttagaacgcggctacaat

SEQ ID NO: 30 - DNA, 30 bp, Artificial Sequence
  Synthetic Construct
taacggcgtgatagggagaccggcagatcc

SEQ ID NO: 31 - DNA, 31 bp, Artificial Sequence
  Synthetic Construct
atacactgcagttatgcaatttatcgcaccc

SEQ ID NO: 32 - DNA, 33 bp, Artificial Sequence
  Synthetic Construct
aatctgctctgatgcggtcgtgaaatgcttcca

SEQ ID NO: 33 - DNA, 32 bp, Artificial Sequence
  Synthetic Construct
cacagaatcaggacaaatggcattcaatgctg

SEQ ID NO: 34 - DNA, 29 bp, Artificial Sequence
  Synthetic Construct
atactgtcgactcaatggcatatgcagcg

SEQ ID NO: 35 - DNA, 40 bp, Artificial Sequence
  Synthetic Construct
aagcatttcacgaccgcatcagagcagattgtactgagag

SEQ ID NO: 36 - DNA, 41 bp, Artificial Sequence
  Synthetic Construct
ttgaatgccatttgtcctgattctgtggataaccgtattac

SEQ ID NO: 37 - DNA, 20 bp, Artificial Sequence
  Synthetic Construct
cggggcgaaagtgattgaga

SEQ ID NO: 38 - DNA, 20 bp, Artificial Sequence
  Synthetic Construct
aattgccgcctgggtattgg

SEQ ID NO: 39 - DNA, 22 bp, Artificial Sequence
  Synthetic Construct
acctttactaccgcactgctgg

SEQ ID NO: 40 - DNA, 22 bp, Artificial Sequence
  Synthetic Construct
gcgggagtcagtgaacaggtac

SEQ ID NO: 41 - DNA, 25 bp, Artificial Sequence
  Synthetic Construct
gatcttgagtccgtaaaacaggctt

SEQ ID NO: 42 - DNA, 22 bp, Artificial Sequence
  Synthetic Construct
ttccgctcaagccattgtagtg

SEQ ID NO: 43 - DNA, 33 bp, Artificial Sequence
  Synthetic Construct
actgagccatggcgaaaatcaataaagtagatc

SEQ ID NO: 44 - DNA, 34 bp, Artificial Sequence
  Synthetic Construct
tgatccgagctcctattattccagtgttcccgcc

SEQ ID NO: 45 - DNA, 32 bp, Artificial Sequence
  Synthetic Construct
actccggaattcatgacgcaatctcgattgca

SEQ ID NO: 46 - DNA, 35 bp, Artificial Sequence
  Synthetic Construct
acctgcgagctcccgttgttccacctgatattatg

SEQ ID NO: 47 - DNA, 34 bp, Artificial Sequence
  Synthetic Construct
gcatagaattcatgacagcacatgaccaggagct

SEQ ID NO: 48 - DNA, 39 bp, Artificial Sequence
  Synthetic Construct
gcatagagctcctacataagtgtccaaattccgacattc

SEQ ID NO: 49 - DNA, 20 bp, Artificial Sequence
  Synthetic Construct
cccgttctggataatgtttt

SEQ ID NO: 50 - DNA, 20 bp, Artificial Sequence
  Synthetic Construct
aaagtcacggttgttattcc

SEQ ID NO: 51 - DNA, 20 bp, Artificial Sequence
  Synthetic Construct
catttaatgccgctcatatt

SEQ ID NO: 52 - DNA, 20 bp, Artificial Sequence
  Synthetic Construct
accgctcaattattgagatt